SEQUENCE LISTING (2 sequences)

SEQ ID NO: 1 - DNA, 18 bp, synthetic construct
  source 1..18: mol_type = other DNA
  misc_feature 1..18: note = bacteriophage T7 RNA polymerase promoter
taatacgactcactatag

SEQ ID NO: 2 - DNA, 29 bp, synthetic construct
  source 1..29: mol_type = other DNA
  misc_feature 1..29: note = T7 terminator sequence
aacccctctctaaacggaggggttttttt

(19) United States
(12) Patent Application Publication
(10) Pub. No.: US 2025/0146038 A1
(43) Pub. Date: May 8, 2025

(54) METHODS FOR GENERATING FUNCTIONAL SELF-REPLICATING RNA MOLECULES

(71) Applicant: Replicate Bioscience, Inc., San Diego, CA (US)

(72) Inventors: Nathaniel Stephen Wang, San Diego, CA (US); Shigeki Joseph MIYAKE-STONER, San Diego, CA (US)

(21) Appl. No.: 18/833,620

(22) PCT Filed: Jan. 27, 2023
(86) PCT No.: PCT/US2023/061489

§ 371 Date: Jul. 26, 2024

Related U.S. Application Data
(60) Provisional application No. 63/303,886, filed on Jan. 27, 2022.

(51) Int. Cl. C12P 19/34; A01K 67/0275; A61K 39/205; A61P 37/00; C12N 15/86

(52) U.S. Cl. CPC C12P 19/34; A01K 67/0275; A61K 39/205; A61P 37/00; C12N 15/86; C12N 2770/36122; C12N 2770/36143; C12N 2770/36151

(57) ABSTRACT

The present disclosure relates generally to methods of generating functional self-replicating RNAs from non-functional positive-sense single-stranded RNA (+ssRNA) virus genomes such as alphavirus genomes or srRNAs. More particularly, the methods pertain to the assembly of a plurality of nucleic acid fragments to generate de novo functional srRNAs. Also provided are compositions, nucleic acid constructs, vectors and recombinant cells including such functional srRNAs. Further disclosed herein are methods for inducing a pharmacodynamic effect in a subject as well as methods for preventing and/or treating a health condition in a subject in need thereof.

### CROSS-REFERENCE TO RELATED APPLICATION

The present application claims priority to U.S. Provisional Patent Application Ser. No. 63/303,886, filed on Jan. 27, 2022. The content of the above-referenced application is herein expressly incorporated by reference in its entirety, including any drawings.

### FIELD

The present disclosure generally relates to the field of molecular virology and immunology. In particular, the present disclosure relates to new methods of generating functional self-replicating RNAs (srRNA), e.g., replicons. More particularly, the disclosure relates to methods of generating functional srRNAs from non-functional positive-sense, single-stranded RNA (+ssRNA) viral genomes or non-functional srRNAs or combinations of functional and non-functional srRNAs. The disclosure also relates to compositions containing such functional srRNAs.

### BACKGROUND

In recent years, several different groups of animal viruses have been subjected to genetic manipulation either by homologous recombination or by direct engineering of their genomes. The availability of reverse genetics systems for both DNA and RNA viruses has created new perspectives for the use of recombinant viruses, for example, as vaccines, expression vectors, anti-tumor agents, gene therapy vectors, and drug delivery vehicles.

Many viral-based expression vectors have been deployed for expression of heterologous proteins in cultured recombinant cells and the application of modified viral vectors for gene expression in host cells continues to expand. Recent advances in this regard include further development of techniques and systems for production of multi-subunit protein complexes, and co-expression of protein-modifying enzymes to improve heterologous protein production. Other recent progresses regarding viral expression vector technologies include many advanced genome engineering applications for controlling gene expression, preparation of viral vectors, in vivo gene therapy applications, and creation of vaccine delivery vectors.

Self-replicating RNA (srRNA) molecules that replicate in host cells can enhance the efficiency of RNA delivery and expression of the encoded gene products by amplifying the amount of RNA encoding a desired gene product. srRNA molecules can be based, for example, on srRNA from alphaviruses. Different alphavirus species and subspecies have evolved unique and diverse mechanism in regard to immunomodulation and gene expression in different hosts and tissues. Their untested properties could have a great impact on human health when employed as srRNA vectors for expression of a desired gene of interest (GOI), such as antigens for vaccination or therapeutic proteins. However, published or deposited sequences of alphavirus genomes are frequently incorrect or incomplete. Even some accurate alphavirus are not adequate to create srRNAs capable of self-replication and gene expression.

There is a need for efficient and cost-effective methods for generating functional srRNAs that can be used for expressing products of interest. There is also a need for methods that generate functional srRNA molecules from non-functional alphavirus genomes or other positive-sense single-stranded RNA viruses.

### SUMMARY

Provided herein, inter alia, are methods for generating functional self-replicating RNAs (srRNAs), e.g., replicons, from positive-sense single-stranded RNA (+ssRNA) viral genomes, such as alphavirus genomes. The functional srRNAs are suitable for expressing molecules of interest such as, for example, vaccines and therapeutic polypeptides. Also provided are functional srRNAs, nucleic acid constructs, vectors, and recombinant cells expressing such srRNA constructs as well as pharmaceutical compositions containing the same. Further disclosed herein are methods for inducing a pharmacodynamic effect in a subject and, in particular, methods for eliciting an immune response and methods for preventing and/or treating a health condition in a subject in need thereof, wherein the methods include prophylactically or therapeutically administering one or more of the functional srRNA, nucleic acid constructs, vectors, recombinant cells, and/or the pharmaceutical composition of the disclosure.

In one aspect of the disclosure, provided herein are methods for generating a functional self-replicating RNA (srRNA), e.g., replicon, the method including: (a) providing one or more positive-sense single-stranded RNA (+ssRNA) viral genomes or non-functional srRNAs, wherein at least one of the one or more +ssRNA viral genomes or srRNAs is non-functional, (b) removing one or more RNA polymerase transcription termination site or cryptic transcription termination site from the one or more +ssRNA viral genomes or srRNAs, (c) generating a plurality of nucleic acid fragments each comprising a nucleotide sequence derived from the one or more +ssRNA viral genomes or srRNAs, and (d) assembling the plurality of nucleic acid fragments to generate a de novo functional srRNA assembly.

Non-limiting exemplary embodiments of the methods of the disclosure can include one or more of the following features. In some embodiments, the one or more RNA polymerase termination sites or cryptic termination sites comprises a bacteriophage T7 termination site or cryptic T7 termination site. In some embodiments, the one or more RNA polymerase termination sites is an SP6 RNA polymerase termination site or an SP6 RNA polymerase cryptic termination site. In some embodiments, the plurality of nucleic acid fragments are each about 60 nucleotides to about 5,000 nucleotides in length. In some embodiments, the plurality of nucleic acid fragments are single-stranded or double-stranded nucleic acids. In some embodiments, the de novo functional srRNA assembly is devoid of at least a portion of the nucleic acid sequence encoding one or more viral structural proteins. In some embodiments, the de novo functional srRNA assembly is devoid of the nucleic acid sequence encoding one or more viral structural proteins. In some embodiments, the de novo functional srRNA assembly is devoid of a substantial portion of the nucleic acid sequence encoding one or more viral structural proteins. In some embodiments, the de novo functional srRNA assembly comprises no nucleic acid sequence encoding viral structural proteins.

In some embodiments, at least one of the one or more +ssRNA viral genomes or srRNAs is of a virus belonging to the Alphavirus genus of the Togaviridae family. In some embodiments, at least one of the one or more +ssRNA viral genomes or srRNAs is of an alphavirus species belonging to VEEV/EEEV group, or the SFV group, or the SINV group. Examples of alphaviruses suitable for the compositions and methods disclosed herein include Eastern equine encephalitis virus (EEEV), Venezuelan equine encephalitis virus (VEEV), Everglades virus (EVEV), Mucambo virus (MUCV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu virus (NDUV), Madariaga virus (MADV), and Buggy Creek virus. In some embodiments, at least one of the +ssRNA viral genomes or srRNAs is of Eastern equine encephalitis virus (EEEV), Chikungunya virus (CHIKV), Sindbis virus (SINV), Venezuelan equine encephalitis virus (VEE), Madariaga virus (MADV), Western equine encephalitis virus (WEEV), or Semliki Forest virus (SFV).

In some embodiments, the methods further include incorporating a nucleic acid sequence encoding a heterologous gene into the de novo srRNA assembly. In some embodiments, the heterologous gene is operably linked to a subgenomic (sg) promoter. In some embodiments, the sg promoter is a 26S subgenomic promoter.

In some embodiments, the methods further include removing one or more restriction enzyme sites from one or more of the +ssRNA genomes or srRNAs. In some embodiments, at least one of the removed restriction enzyme sites is recognized by a restriction enzyme suitable for linearization of the de novo srRNA assembly or for insertion of the heterologous gene into the de novo srRNA assembly. In some embodiments, the generated functional srRNA assembly comprises a 3′ polyadenylate tract (poly(A) tail). In some embodiments, the 3′ poly(A) tail comprises at least 11 adeninosine nucleotides.

In some embodiments, the methods further include replacing one or more untranslated regions (UTR), or a portion thereof, in the de novo srRNA assembly with a UTR from a different species or subspecies of the +ssRNA viral genome. In some embodiments, the UTR, or portion thereof, is derived from another strain of the same +ssRNA virus species. In some embodiments, the UTR or portion thereof is a 3′ UTR, a 5′ UTR, or a portion of any thereof. In some embodiments, the methods further include selecting the UTR from a virulent species or an avirulent species of a +ssRNA virus.

In some embodiments, the methods further include replacing a non-structural protein (nsP), or a portion thereof, in the de novo srRNA assembly with a heterologous nsP. In some embodiments, the heterologous nsP or portion thereof is from another +ssRNA virus species or subspecies. In some embodiments, the nsP or portion thereof is derived from another strain of the same +ssRNA virus species. In some embodiments, the nsP or portion thereof is nsP1, nsP2, nsP3, nsP4, or a portion of any thereof. In some embodiments, the methods further include selecting the nsP or a UTR from a virulent species or an avirulent species of a +ssRNA virus.

In some embodiments, the methods further include assessing functionality of the de novo srRNA assembly. In some embodiments, the assessing functionality is carried out in vitro, in vivo, and/or ex vivo. In some embodiments, the assessing functionality comprises analyzing the de novo srRNA assembly for capability of self-replicating in vivo and/or ex vivo. In some embodiments, the assessing functionality comprises an assay selected from the group consisting of: detection of RNA replication, detection of viral protein expression, detection of cytopathic effect (CPE), and detection of heterologous gene expression. In some embodiments, the assessing functionality of the de novo srRNA assembly does not include incorporating a nucleic acid sequence encoding a heterologous gene into the de novo srRNA assembly.

In one aspect of the disclosure, provided herein are functional self-replicating RNAs (srRNAs) generated by a method as disclosed herein, wherein the functional srRNAs include a heterologous UTR and/or a heterologous nsP.

In another aspect of the disclosure, provided herein are nucleic acid constructs encoding a srRNA as disclosed herein. In a related aspect, provided herein are vectors including a nucleic acid construct as disclosed herein.

In another aspect, provided herein are recombinant cells including: (a) a functional srRNA as disclosed herein, (b) a nucleic acid construct as disclosed herein or (c) a vector as disclosed herein. Non-limiting exemplary embodiments of the recombinant cells of the disclosure can include one or more of the following features. In some embodiments, the recombinant cell is a eukaryotic cell. In some embodiments, the recombinant cell is an animal cell. In some embodiments, the recombinant cell is a vertebrate animal cell or an invertebrate animal cell. In some embodiments, the recombinant cell is an insect cell. In some embodiments, the recombinant cell is a mosquito cell. In some embodiments, the recombinant cell is a mammalian cell. In some embodiments, the recombinant cell is selected from the group consisting of a monkey kidney CV1 cell transformed by SV40 (COS-7), a human embryonic kidney cell (e.g., HEK 293 or HEK 293 cell), a baby hamster kidney cell (BHK), a mouse sertoli cell (e.g., TM4 cells), a monkey kidney cell (e.g., CV1), a human cervical carcinoma cell (e.g., HeLa), canine kidney cell (e.g., MDCK), buffalo rat liver cell (e.g., BRL 3A), human lung cell (e.g., W138), human liver cell (e.g., Hep G2), mouse mammary tumor (e.g., MMT 060562), TRI cell, FS4 cell, a Chinese hamster ovary cell (CHO cell), an African green monkey kidney cell (e.g., Vero cell), a human A549 cell, a human cervix cell, a human CHME5 cell, a human PER.C6 cell, a NS0 murine myeloma cell, a human epidermoid larynx cell, a human fibroblast cell, a human HUH-7 cell, a human MRC-5 cell, a human muscle cell, a human endothelial cell, a human astrocyte cell, a human macrophage cell, a human RAW 264.7 cell, a mouse 3T3 cell, a mouse L929 cell, a mouse connective tissue cell, a mouse muscle cell, and a rabbit kidney cell.

In another aspect, provided herein are pharmaceutical compositions including: (a) a functional srRNA as disclosed herein, (b) a nucleic acid construct as disclosed herein, (c) a vector as disclosed herein, (c) a recombinant cell as disclosed herein.

Non-limiting exemplary embodiments of the pharmaceutical compositions of the disclosure can include one or more of the following features. In some embodiments, the pharmaceutical compositions include a functional srRNA as disclosed herein, and a pharmaceutically acceptable excipient. In some embodiments, the pharmaceutical compositions include a nucleic acid construct as disclosed herein and a pharmaceutically acceptable excipient. In some embodiments, the pharmaceutical compositions include a vector as disclosed herein and a pharmaceutically acceptable excipient. In some embodiments, the pharmaceutical compositions include a recombinant cell as disclosed herein and a pharmaceutically acceptable excipient. In some embodiments, the pharmaceutical composition is formulated in a liposome, a lipid-based nanoparticle (LNP), a polymer nanoparticle, a polyplex, a viral replicon particle (VRP), a microsphere, an immune stimulating complex (ISCOM), a conjugate of a bioactive ligand, or a combination of any thereof. In some embodiments, the pharmaceutical composition is an immunogenic composition. In some embodiments, the immunogenic composition is formulated as a vaccine. In some embodiments, the composition is substantially non-immunogenic to a subject. In some embodiments, the pharmaceutical composition is formulated as an adjuvant. In some embodiments, the pharmaceutical composition is formulated for one or more of intranasal administration, transdermal administration, intraperitoneal administration, intramuscular administration, intratracheal administration, intranodal administration, intratumoral administration, intraarticular administration, intravenous administration, subcutaneous administration, intravaginal administration, intrathecal administration, intraocular, rectal, and oral administration.

In another aspect, provided herein are kits for the practice of a method disclosed herein. In some embodiments, the kits include: (a) a functional srRNA as disclosed herein, (b) a nucleic acid construct as disclosed herein, (c) a vector as disclosed herein, (d) a recombinant cell as disclosed herein and/or (e) a pharmaceutical composition as disclosed herein.

In another aspect, provided herein are transgenic animals including: (a) a functional srRNA a as disclosed herein, (b) a nucleic acid construct as disclosed herein, (c) a vector as disclosed herein, and/or (d) a recombinant cell as disclosed herein. Non-limiting exemplary embodiments of the transgenic animals of the disclosure can include one or more of the following features. In some embodiments, the animal is a vertebrate animal or an invertebrate animal. In some embodiments, the animal is an insect. In some embodiments, the animal is a mammal. In some embodiments, the mammal is a non-human mammal.

In another aspect, provided herein are methods for producing a polypeptide of interest, the methods including (i) rearing a transgenic animal as disclosed herein, or (ii) culturing a recombinant cell including a nucleic acid construct as disclosed herein under conditions wherein the recombinant cell produces the polypeptide encoded by the srRNA.

In another aspect, provided herein are methods for inducing a pharmacodynamic effect in a subject, the methods include administering to the subject a composition including: (a) a functional srRNA as disclosed herein, (b) a nucleic acid construct as disclosed herein, (c) a recombinant cell as disclosed herein, and/or (d) a pharmaceutical composition as disclosed herein. In some embodiments, the pharmacodynamic effect includes eliciting an immune response in the subject.

In another aspect, provided herein are methods for preventing or treating a health condition in a subject, the method includes administering to the subject a composition including: (a) a functional srRNA as disclosed herein, (b) a nucleic acid construct as disclosed herein, (c) a recombinant cell as disclosed herein, and/or (d) a pharmaceutical composition as disclosed herein. In some embodiments, the pharmacodynamic effect includes eliciting an immune response in the subject.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1A is a graphical representation of an example of an alphavirus and a deposited sequence. The alphavirus has a 5′ UTR, nucleic acids encoding four nonstructural proteins nsP1, nsP2, nsP3, nsP4, a 26S subgenomic promoter, a structural polyprotein and a 3′ UTR.

FIG. 1B is a graphical illustration of an exemplary method in accordance with some embodiments of the disclosure in which nucleic acid fragments (synthesized de novo or derived from a viral genome or srRNA or combination thereof) are assembled into a larger construct. The self-replicating assembly or construct, as shown, includes an RNA polymerase promoter, a 5′ UTR, encodes four nonstructural proteins, nsP1, nsP2, nsP3, nsP4, a 26S subgenomic promoter, an adaptor sequence and/or transgene, a 3′ UTR and a polyadenylate tract (pA) followed by a terminator and/or restriction site. This construct can be used as the template to generate srRNA for functional testing in vitro and/or in vivo.

FIG. 2 is a graphical illustration of an exemplary method in accordance with some embodiments of the present disclosure. Swapping one or more UTRs from a different species, subspecies, or strain can be a step for functionalizing a self-replicating RNA molecule. Similarly, swapping one or more NSPs from a different species, subspecies, or strain can be a step for functionalizing a self-replicating RNA molecule.

FIG. 3 is a graphical representation of four non-limiting examples of the modified alphavirus genome designs in accordance with some embodiments of the disclosure, in which the nucleic acid sequence encoding viral structural proteins of the original virus have been completely deleted. Non-structural proteins nsP1, nsP2, nsP3, and nsP4 are shown. A non-limiting example of a modified CHIKV design can be based on CHIKV strain S27 and further can contain a heterologous gene (GOI) placed under control of a 26S subgenomic promoter. A non-limiting example of a modified CHIKV design can also be based on CHIKV strain DRDE-06, contains the 3′ UTR derived from the CHIKV strain S27, and further can contain a heterologous gene (GOI) placed under control of a 26S subgenomic promoter. A non-limiting example of a modified SINV design can be based on SINV strain Girdwood and further can contain a heterologous gene (GOI) placed under control of a 26S subgenomic promoter. A non-limiting example of a modified SINV design can be based on SINV strain AR86, contains nsP2 derived from the SINV strain Girdwood, and further can contain a heterologous gene (GOI) placed under control of a 26S subgenomic promoter.

FIG. 4 is a graphical illustration of an exemplary alphavirus-based srRNA design pRB_017 CHIKV-DRDE-S27-HPV16 construct in accordance with some embodiments of the disclosure, in which the sequences encoding the modified CHIKV DRDE-06 is incorporated into expression vectors, which also include coding sequences for an exemplary gene of interest (GOI), e.g., human papillomavirus (HPV) oncoproteins E6/E7.

FIG. 5 is a graphical illustration of an exemplary alphavirus-based srRNA design CHIKV-DRDE-HA construct in accordance with some embodiments of the disclosure, in which the sequence encoding the modified CHIKV DRDE-06 genome is incorporated into expression vectors, which also include coding sequences for an exemplary gene of interest (GOI), e.g., hemagglutinin precursor (HA) of the influenza A virus H5N1.

FIG. 6 is a graphical illustration of an exemplary alphavirus-based srRNA design CHIKV-DRDE-Oncology construct in accordance with some embodiments of the disclosure, in which the sequence encoding the modified CHIKV DRDE-06 genome is incorporated into expression vectors, which also include coding sequences for an exemplary gene of interest (GOI), e.g., a synthetic sequence cassette encoding genes or parts of genes relevant to oncology (Estrogen Receptor 1 (ESR1), Human Epidermal Growth Factor Receptor 2 (HER2), and Human Epidermal Growth Factor Receptor 3 (HER3)).

FIG. 7 graphically summarizes the results of experiments performed to demonstrate that a non-functional alphavirus genome or srRNA can be functionalized by replacing a defective nsP sequence with a corresponding functional nsP derived from a heterologous alphavirus genome or srRNA. This figure depicts contour plots of BHK-21 cells which have been transformed with exemplary alphavirus genome designs in accordance with some embodiments of the disclosure. In these experiments, the alphavirus genome designs were each introduced into BHK-21 cells by electroporation, and 20 hours following transformation, the cells were fixed and permeabilized and stained using a PE-conjugated anti-double stranded RNA (dsRNA) mouse monoclonal antibody (J2, Scicons) to quantify the frequency of dsRNA+ cells by fluorescence flow cytometry. The ability of the alphavirus genome designs to undergo RNA replication to result in production of dsRNA is indicated.

FIGS. 8A-8B are bar charts illustrating in vivo immunogenicity of a panel of srRNAs that were functionalized in accordance with a method disclosed herein. This panel of functionalized srRNAs encoded an exemplary viral antigen, which is an envelope glycoprotein G of a rabies virus (RABV-G). The panel included srRNAs derived from Venezuelan equine encephalitis virus (VEE.TC83), Chikungunya virus strains S27 (CHIK.S27) and DRDE-06 (CHIK.DRDE), Sindbis virus strains Girdwood (SIN.GW) and AR86-Girdwood Hybrid 1 (SIN.AR86), and Eastern equine encephalitis virus (EEE.FL93). FIG. 8A shows the quantification of antigen-specific splenic T cell responses evaluated by ELISpot after two immunizations. FIG. 8B shows anti-rabies neutralizing antibody titers from sera after two immunizations.

FIG. 9 graphically illustrates that antigen-specific T cell responses can be detected from functionalized srRNA vectors encoding infectious disease-associated targets (Day 14 post-prime with each vector). In vivo administration of the functionalized CHIKV- and SINV-derived vectors encoding HA antigen from H5N1 in BALB/c mice generates antigen-specific CD4+ and CD8+ T cell and functional antibody responses. Geometric mean with geometric SD. One-way ANOVA.

FIG. 10 graphically illustrates that antigen-specific T cell responses can be detected from functionalized srRNA vectors encoding oncology-associated targets (Day 14 post-prime with each vector). In vivo administration of the CHIKV- and SINV-derived vectors encoding activating mutations from ESR1 and PI3K alongside truncated HER2 and kinase-dead HER3 proteins in BALB/c mice generate robust T cell responses. Geometric mean with geometric SD. One-way ANOVA.

### DETAILED DESCRIPTION OF THE DISCLOSURE

Provided herein, inter alia, are methods for generating functional self-replicating RNAs (srRNAs) from one or more non-functional positive-sense single-stranded viral RNA genomes, such as alphavirus genomes, or non-functional self-replicating RNAs. The functional srRNAs are suitable for expressing molecules of interest such as, for example, vaccines and therapeutic polypeptides. Also provided herein are nucleic acid constructs including nucleic acids encoding srRNA, and vectors containing the srRNAs of the present disclosure. Further provided are self-replicating RNAs that are de novo assembled from a plurality of nucleic acid fragments of one or more non-functional alphavirus genome, single-stranded RNA (ssRNA) genomes, and/or srRNAs.

As discussed above, there is a need in the art for efficient and cost-effective methods for generating functional srRNAs, particularly those that can be used for expressing products of interest. Publicly available alphavirus genomic data does not always provide nucleotide sequences that are capable of having the nucleic acid sequences encoding the structural proteins directly replaced with a gene of interest (GOI) to result in self-replicating and transgene-expressing srRNAs. In particular, a significant number of publicly available alphavirus genomes were found to be non-functional, e.g., incapable of undergoing replication and/or expressing a transgene.

Functional srRNAs based on alphaviruses can be used as robust expression systems. As used herein, a functional srRNA is an srRNA capable of undergoing replication and/or expressing a transgene. For example, it has been reported that an advantage of using alphaviruses as viral expression vectors is that they can direct the synthesis of large amounts of heterologous proteins in recombinant host cells. Among other advantages, polypeptides such as therapeutic single chain antibodies can be most effective if expressed at high levels in vivo. In addition, for producing recombinant antibodies purified from cells in culture (ex vivo), high protein expression from a srRNA can increase overall yields of the antibody product. Furthermore, if the protein being expressed is a vaccine antigen, high level expression can induce the most robust immune response in vivo.

It has not been fully appreciated that full-length viruses and synthetic srRNAs do not have the same capacity for replication. In particular, many full-length viruses and srRNA from publicly available resources are functionally defective as srRNAs. Currently, a modification approach to functionalize a defective alphavirus genome or srRNA is to revert one or more key point mutations related to virulence that diverged between strains, for example mutations that diverged between a functional strain (e.g., Girdwood) and a non-functional strain (e.g., AR86). However, this strategy often fails or a solution is arrived at arbitrarily, indicating that there is far more uncharacterized and thus unpredictable sequence divergence between strains. Therefore, there is a need for a more rapid and efficient way to identify functional alphavirus strains (instead of simply reverting point mutations or selecting arbitrary regions to generate chimeras). There is also a need for a reliable method to generate functional srRNAs from non-functional viral genomes or/and non-functional srRNAs.

Given the differential presence of host cell attenuating factors in non-structural and structural regions of Alphaviruses, deleting the structural genes to allow for heterologous gene expression in synthetic vectors will have varied impacts on individual vectors. Synthetic srRNA with different host attenuating factors in the non-structural regions will differentially excel at the induction of immune responses to heterologous genes that are expressed.

As described in greater detail, provided herein, inter alia, are new methods useful for generating a functional srRNA from one or more of a defective (e.g., non-functional) alphavirus genome or positive-sense single stranded RNA (+ssRNA) viruses or from a combination of functional and non-functional srRNAs or fragments thereof.

Headings, e.g., (a), (b), (i) etc., are presented merely for ease of reading the specification and claims. The use of headings in the specification or claims does not require the steps or elements be performed in alphabetical or numerical order or the order in which they are presented.

### I. General Techniques

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

### II. Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges can independently be included in the smaller ranges, and are also encompassed within the disclosure, subject to any specifically excluded limit in the stated range. Where the stated range includes one or both of the limits, ranges excluding either or both of those included limits are also included in the disclosure.

The terms “administration” and any grammatical variation thereof, as used herein, refer to the delivery of a bioactive composition or formulation by an administration route comprising, but not limited to, intranasal, transdermal, intravenous, intra-arterial, intramuscular, intranodal, intraperitoneal, subcutaneous, intramuscular, oral, intravaginal, and topical, intrathecal, intraocular, rectal administration or combinations thereof. The term includes, but is not limited to, administering by a medical professional and self-administering.

The term “derived from” as used herein refers to the source (e.g., naturally occurring nucleic acid sequence of a virus) from which a nucleic acid or polypeptide sequence is either obtained (e.g., isolated from) or is designed (i.e., engineered). The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications can occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

The term “construct” refers to a recombinant molecule, e.g., recombinant nucleic acid or polypeptide, including one or more nucleic acid sequences or amino acid sequences from heterologous sources. For example, polypeptide constructs can be chimeric polypeptide molecules in which two or more amino acid sequences of different origin are operably linked to one another in a single polypeptide construct. Similarly, nucleic acid constructs can be chimeric nucleic acid molecules in which two or more nucleic acid sequences of different origin are assembled into a single nucleic acid molecule. Representative nucleic acid constructs can include any recombinant nucleic acid molecules, linear or circular, single stranded or double stranded DNA or RNA nucleic acid molecules, derived from any source, such as a plasmid, cosmid, virus, autonomously replicating polynucleotide molecule, phage, capable of genomic integration or autonomous replication, comprising a nucleic acid molecule where one or more nucleic acid sequences have been operably linked. In some embodiments, one or more nucleic acid constructs can be incorporated (e.g., inserted) within a single nucleic acid molecule, such as a single vector, or can be incorporated (e.g., inserted) within two or more separate nucleic acid molecules, such as two or more separate vectors. The term “vector” is used herein to refer to a nucleic acid molecule or sequence capable of transferring or transporting another nucleic acid molecule. Thus, the term “vector” encompasses both DNA-based vectors and RNA-based vectors. The term “vector” includes cloning vectors and expression vectors, as well as viral vectors and integrating vectors. An “expression vector” is a vector that includes a regulatory region, thereby capable of expressing DNA sequences and fragments in vitro, ex vivo, and/or in vivo. In some embodiments, a vector may include sequences that direct autonomous replication in a cell such as, for example a plasmid (DNA-based vector) or a self-replicating RNA vector. In some embodiments, a vector may include sequences sufficient to allow integration into host cell DNA. In some embodiments, a vector may include DNA sequences that can be transcribed into RNA in vitro and/or in vivo. Useful vectors include, for example, plasmids (e.g., DNA plasmids or RNA plasmids), transposons, cosmids, bacterial artificial chromosomes, and viral vectors. In some embodiments, the vector of the disclosure can be single-stranded vector (e.g., ssDNA or ssRNA). In some embodiments, the vector of the disclosure can be double-stranded vector (e.g., dsDNA or dsRNA). In some embodiments, a vector is a gene delivery vector. In some embodiments, a vector is used as a gene delivery vehicle to transfer a gene into a cell. In some embodiments, the vector of the disclosure is a self-replicating RNA (srRNA) vector.

The term “effective amount”, “therapeutically effective amount”, or “pharmaceutically effective amount” of a composition of the disclosure, e.g., nucleic acid constructs, recombinant cells, recombinant polypeptides, and/or pharmaceutical compositions, generally refers to an amount sufficient for the composition to accomplish a stated purpose relative to the absence of the composition (e.g., achieve the effect for which it is administered, stimulate an immune response, prevent or treat a disease, or reduce one or more symptoms of a disease, disorder, infection, or health condition). An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “operably linked”, as used herein, denotes a physical or functional linkage between two or more elements, e.g., polypeptide sequences or polynucleotide sequences, which permits them to operate in their intended fashion. For example, the term “operably linked” when used in context of the nucleic acid molecules described herein or the coding sequences and promoter sequences in a nucleic acid molecule means that the coding sequences and promoter sequences are in-frame and in proper spatial and distance away to permit the effects of the respective binding by transcription factors or RNA polymerase on transcription. It should be understood that operably linked elements can be contiguous or non-contiguous (e.g., linked to one another through a linker). Operably linked segments, portions, regions, and domains of the nucleic acid molecules disclosed herein can be contiguous or non-contiguous (e.g., linked to one another through a linker).

The term “cryptic termination site” as used herein refers to a RNA polymerase termination site (e.g., T7 RNAP Tφ terminator) that leads to early transcription termination, and can beis produced from an intergenic or intragenic regions (i.e., is not associated with a gene).

The term “pharmaceutically acceptable excipient” as used herein refers to any suitable substance that provides a pharmaceutically acceptable carrier, additive, or diluent for administration of a compound(s) of interest to a subject. As such, “pharmaceutically acceptable excipient” can encompass substances referred to as pharmaceutically acceptable diluents, pharmaceutically acceptable additives, and pharmaceutically acceptable carriers. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics and additional therapeutic agents) can also be incorporated into the compositions.

The term “portion” as used herein refers to a fraction. With respect to a particular structure such as a polynucleotide sequence or an amino acid sequence or protein the term “portion” thereof may designate a continuous or a discontinuous fraction of said structure. For example, a portion of an amino acid sequence comprises at least 1%, at least 5%, at least 10%, at least 20%, at least 30%, at least 40%, at least 50%, at least 60%, at least 70%, at least 80%, and at least 90% of the amino acids of said amino acid sequence. In addition or alternatively, if the portion is a discontinuous fraction, said discontinuous fraction is composed of 2, 3, 4, 5, 6, 7, 8, or more parts of a structure (e.g., domains of a protein), each part being a continuous element of the structure. For example, a discontinuous fraction of an amino acid sequence may be composed of 2, 3, 4, 5, 6, 7, 8, or more, for example not more than 4 parts of said amino acid sequence, wherein each part comprises at least 1, at least 2, at least 3, at least 4, at least 5 continuous amino acids, at least 10 continuous amino acids, at least 20 continuous amino acids, or at least 30 continuous amino acids of the amino acid sequence.

The term “recombinant” when used with reference to a cell, a nucleic acid, a protein, or a vector, indicates that the cell, nucleic acid, protein or vector has been altered or produced through human intervention such as, for example, has been modified by or is the result of laboratory methods. Thus, for example, recombinant proteins and nucleic acids include proteins and nucleic acids produced by laboratory methods. Recombinant proteins can include amino acid residues not found within the native (non-recombinant or wild-type) form of the protein or can be include amino acid residues that have been modified, e.g., labeled. The term can include any modifications to the peptide, protein, or nucleic acid sequence. Such modifications may include the following: any chemical modifications of the peptide, protein or nucleic acid sequence, including of one or more amino acids, deoxyribonucleotides, or ribonucleotides; addition, deletion, and/or substitution of one or more of amino acids in the peptide or protein; creation of a fusion protein, e.g., a fusion protein comprising an antibody fragment; and addition, deletion, and/or substitution of one or more of nucleic acids in the nucleic acid sequence. The term “recombinant” when used in reference to a cell is not intended to include naturally-occurring cells but encompass cells that have been engineered/modified to include or express a polypeptide or nucleic acid that would not be present in the cell if it was not engineered/modified.

As used herein, a “subject” or an “individual” includes animals, such as human (e.g., human individuals) and non-human animals. In some embodiments, a “subject” or “individual” is a patient under the care of a physician. Thus, the subject can be a human patient or an individual who has, is at risk of having, or is suspected of having a health condition of interest (e.g., cancer or infection) and/or one or more symptoms of the health condition. The subject can also be an individual who is diagnosed with a risk of the health condition and/or disease of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges can independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about” which, as used herein, has its ordinary meaning of approximately. The term “about” is used to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value±up to 10%, up to ±5%, or up to ±1%.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination. All combinations of the embodiments pertaining to the disclosure are specifically embraced by the present disclosure and are disclosed herein just as if each and every combination was individually and explicitly disclosed. In addition, all subcombinations of the various embodiments and elements thereof are also specifically embraced by the present disclosure and are disclosed herein just as if each and every such subcombination was individually and explicitly disclosed herein.

### Positive-Sense Single-Stranded RNA Viruses

Positive-sense single-stranded RNA (+ssRNA) viruses include major pathogens of humans, animals, insects and plants. These viruses have similar genome features and several conserved protein domains.

Eight families of +ssRNA viruses whose members infect vertebrates are currently known. These include three families characterized by non-enveloped capsids, (Hepeviridae, Caliciviridae and Picornaviridae) and four families that have enveloped capsids, (Togaviridae, Arteriviridae, Flaviviridae and Coronaviridae.) These viruses use their genomes as messenger RNA which is translated into one or more polyproteins that are subsequently cleaved, by viral or cellular proteases, into individual proteins. The genomes of these viruses encode an RNA-dependent RNA polymerase which transcribes the positive RNA strand as well as complementary negative RNA strands arising as intermediate products of genome replication. The classification into the different taxonomic families depends on the number of different polyproteins that are synthesized during viral infection as well as the number, size, position and orientation of viral genes in the RNA molecule and the existence of an envelope as a virion component.

The Coronaviridae family of +ssRNA viruses includes the “alphaviruses supergroup.” Alphaviruses are small, enveloped RNA viruses with a single-stranded, positive-sense RNA genome. The alphavirus genus includes, inter alia, the Eastern equine encephalitis virus (EEEV), Venezuelan equine encephalitis virus (VEEV), Chikungunya virus (CHIKV), Sindbis virus (SINV), Madariaga virus (MADV), Semliki Forest virus (SFV), Western equine encephalitis virus (WEEV), or Semliki Forest virus (SFV), Everglades virus (EVEV), Mucambo virus (MUCV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV, Highland J virus (HJV), Fort Morgan virus (FMV), Ndumu virus (NDUV), Madariaga virus (MADV), and Buggy Creek virus, which are all closely related and are able to infect various vertebrates such as mammalians, rodents, fish, avian species, and larger mammals such as humans and horses as well as invertebrates such as insects. In particular, the Sindbis and the Semliki Forest viruses have been widely studied and the life cycle, mode of replication, etc., of these viruses are fairly well characterized.

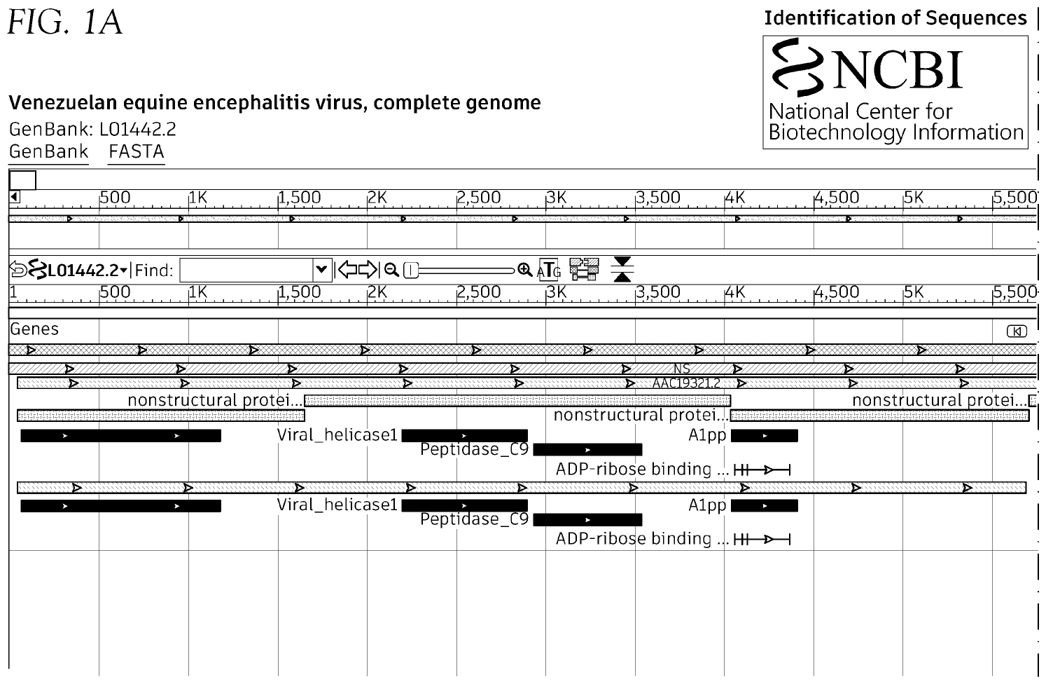

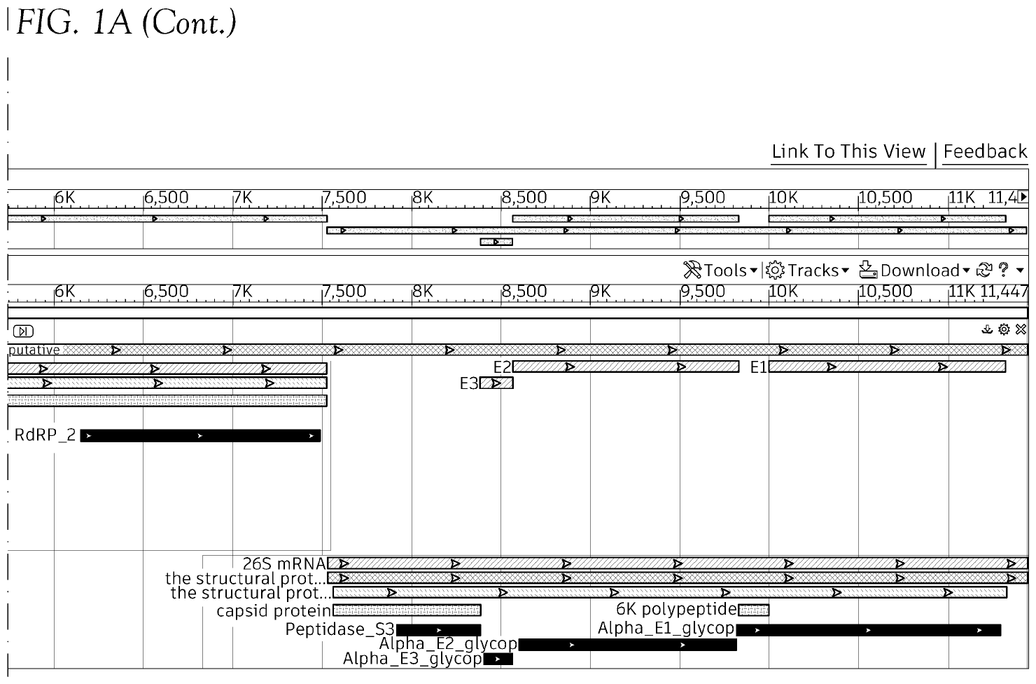

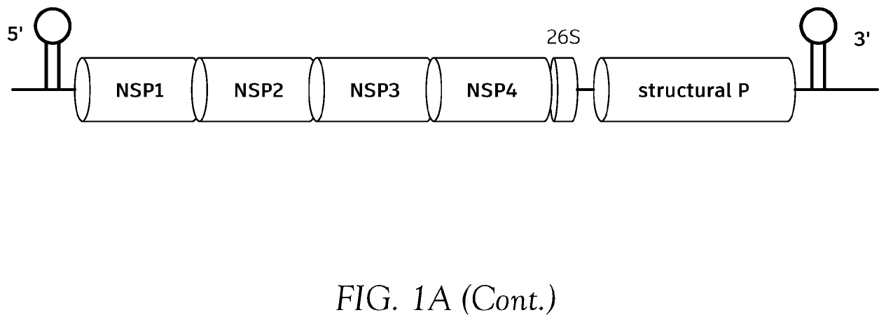

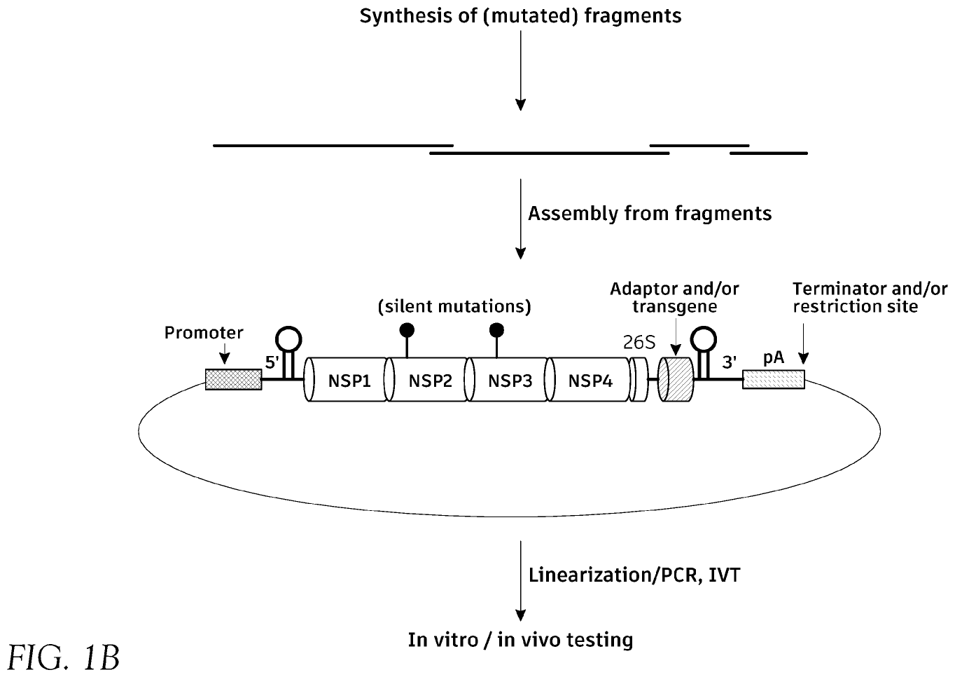

The alphavirus genome is approximately 11-12 kb long, comprising a 5′ prime cap, a 3′ poly(A) tail, and two open reading frames (ORFs): a 4 kb frame encoding the structural polyprotein and a 7 kb frame encoding the nonstructural proteins (nsPs). The 4 kb frame encodes the viral structural proteins such as the capsid protein CP, E1 glycoprotein, E2 glycoprotein, E3 protein, and 6K protein. The non-structural polyprotein (nsP) is cleaved into four different proteins (nsP1, nsP2, nsP3, and nsP4) which are necessary for the transcription and translation of viral mRNA inside the cytoplasm of host cells. A graphical representation of an example of an alphavirus and its structural organization is shown in FIG. 1A.

The nsP1 protein is an mRNA capping enzyme that possesses both guanine-7-methyltransferase (MTase) and guanylyltransferase (GTase) activities, where they direct the methylation and capping of newly synthesized viral genomic and subgenomic RNAs. The MTase motif in the N-terminal domain of nsP1 catalyzes the transfer of the methyl group from S-adenosylmethionine (AdoMet) to the N7 position of a GTP molecule (m7Gppp). GTase then binds the m7Gppp, forming a covalent link with a catalytic histidine (m7Gp-GTase) and releasing PPi. The GTase then transfers the m7Gp molecule to the 5′-diphosphate RNA to create m7GpppNp-RNA. The resulting cap structure is essential for viral mRNA translation and prevents the mRNA from being degraded by cellular 5′ exonucleases. Following the N-terminal domain are features that allow the association of the nsP1 protein to cellular membranes. The presence of α-helical amphipathic loop and palmitoylation sites allow the nsP1 protein and nsP1-containing replication complex to anchor onto the plasma membrane, possibly through nsP1 interaction with the membrane's anionic phospholipids.

The nsP2 protein possesses numerous enzymatic activities and functional roles. The N-terminal region contains a helicase domain that has seven signature motifs of Superfamily 1 (SF1) helicases. It functions as an RNA triphosphatase that performs the first of the viral RNA capping reactions. It also functions as a nucleotide triphosphatase (NTPase), fueling the RNA helicase activity. The C-terminal region of nsP2 contains a papain-like cysteine protease, which is responsible for processing the viral non-structural polyprotein. The protease recognizes conserved motifs within the polyprotein. This proteolytic function is highly regulated and is modulated by other domains of nsP2. The alphavirus nsP2 protein has also been described as a virulence factor responsible for the transcriptional and translational shutoff in infected host cells and the inhibition of interferon (IFN) mediated antiviral responses contributing to the controlling of translational machinery by viral factors.

The precise role(s) of alphavirus nsP3 protein in the replication complex is less clear. The nsP3 protein has three recognized domains: the N-terminal macrodomain with phosphatase activity and nucleic acid binding ability, the alphavirus unique domain (AUD) and the C-terminal hypervariable domain. It has been demonstrated that the deletion of this domain in SFV nsP3 resulted in low viral pathogenicity, suggesting its importance in viral RNA transcription regulation.

The nsP4 polymerase is the most highly conserved protein in alphaviruses, with the most divergent being>50% identical in amino acid sequence when compared with other alphaviral nsP4s. The nsP4 contains the core RNA-dependent RNA polymerase (RdRp) domain at the C-terminal end, determined to be solely responsible for the RNA synthetic properties of the viral replication complex. The RdRp participates in replicating the genomic RNA via a negative strand RNA and transcribing the 26S subgenomic RNA. The N-terminal domain is alphavirus-specific and can be partially disordered structurally.

The 5′ two-thirds of the alphavirus genome encodes a number of nonstructural proteins (nsPs) necessary for transcription and replication of viral RNA. These proteins are translated directly from the RNA and together with cellular proteins form the RNA-dependent RNA polymerase essential for viral genome replication and transcription of subgenomic RNA. Four nonstructural proteins (nsP1, nsP2, nsP3, nsP4) are produced as a single polyprotein constitute the virus' replication machinery. The processing of the polyprotein occurs in a highly regulated manner, with cleavage at the P2/3 junction influencing RNA template use during genome replication. This site is located at the base of a narrow cleft and is not readily accessible. Once cleaved, nsP3 creates a ring structure that encircles nsP2. These two proteins have an extensive interface. Mutations in nsP2 that produce noncytopathic viruses or a temperature sensitive phenotypes cluster at the P2/P3 interface region. P3 mutations opposite the location of the nsP2 noncytopathic mutations prevent efficient cleavage of P2/3. This in turn can affect RNA infectivity altering viral RNA production levels.

The 3′ one-third of the genome comprises subgenomic RNA which serves as a template for translation of all the structural proteins required for forming viral particles (e.g., the core nucleocapsid protein C, and the envelope proteins P62 and E1 that associate as a heterodimer). The viral membrane-anchored surface glycoproteins are responsible for receptor recognition and entry into target cells through membrane fusion. The subgenomic RNA is transcribed from the 26S subgenomic promoter present at the 3′ end of the RNA sequence encoding the nsP4 protein. The proteolytic maturation of P62 into E2 and E3 causes a change in the viral surface. Together the E1, E2, and sometimes E3, glycoprotein “spikes” form an E1/E2 dimer or an E1/E2/E3 trimer, where E2 extends from the center to the vertices, E1 fills the space between the vertices, and E3, if present, is at the distal end of the spike. Upon exposure of the virus to the acidity of the endosome, E1 dissociates from E2 to form an E1 homotrimer, which is necessary for the fusion step to drive the cellular and viral membranes together. The alphaviral glycoprotein E1 is a class II viral fusion protein, which is structurally different from the class I fusion proteins found in influenza virus and HIV. The E2 glycoprotein functions to interact with the nucleocapsid through its cytoplasmic domain, while its ectodomain is responsible for binding a cellular receptor. Most alphaviruses lose the peripheral protein E3, while in Semliki viruses it remains associated with the viral surface.

Alphavirus replication has been reported to take place on membranous surfaces within the host cell. In the first step of the infectious cycle, the 5′ end of the genomic RNA is translated into a polyprotein (nsP1-4) with RNA polymerase activity that produces a negative strand complementary to the genomic RNA. In a second step, the negative strand is used as a template for the production of two RNAs, respectively: (1) a positive genomic RNA corresponding to the genome of the secondary viruses producing, by translation, other nsP and acting as a genome for the virus; and (2) subgenomic RNA encoding the structural proteins of the virus forming the infectious particles. The positive genomic RNA/subgenomic RNA ratio is regulated by proteolytic autocleavage of the polyprotein to nsP1, nsP2, nsP3 and nsP4. In practice, the viral gene expression takes place in two phases. In a first phase, there is main synthesis of positive genomic strands and of negative strands. During the second phase, the synthesis of subgenomic RNA is virtually exclusive, thus resulting in the production of large amount of structural protein.

Alphaviruses utilize motifs contained in their UTRs, structural regions, and nonstructural regions to impact their replication in host cells. These regions also contain mechanism to evade host cell innate immunity. However, significant differences among alphavirus species have been reported. For example, New World and Old World Alphaviruses have evolved different components to exploit stress granules, JAK-STAT signaling, FXR, and G3BP proteins within cells for assembly of viral replication complexes. Which part of the genome contains these components also varies between Alphaviruses. For example, bypassing activation of PKR and subsequent phosphorylation of EIF2alpha is done via the downstream loop (DLP) in some Old World Alphaviruses such as Sindbis, but bypassing this pathway is believed to be done via NSP4 in Chikungunya, which lacks a recognizable DLP. In addition, beyond variation between individual Alphaviruses, there are often differences within strains of Alphaviruses as well that can account for changes in characteristics such as virulence. For example, sequence variations between North American and South American strains of Eastern Equine Encephalitis Virus (EEEV) alter the ability to modulate the STAT1 pathway leading to differential induction of Type I interferons and resulting changes in virulence.

### Self-Replicating RNA

As will be appreciated by the skilled artisan, the term “self-replicating RNA” refers to RNA molecule that contains all of the genetic information required for directing its own amplification or self-replication within a permissive cell. Therefore, srRNA is sometimes also referred to as “self-amplifying RNA” (saRNA), a term that encompasses “replicon” or “replicon RNA” or “RNA replicon.” To direct its own replication, the srRNA generally (1) encodes polymerase, replicase, or other proteins which may interact with viral or host cell-derived proteins, nucleic acids or ribonucleoproteins to catalyze the RNA amplification process; and (2) contain cis-acting RNA sequences required for replication and transcription of the subgenomic replicon-encoded RNA. These sequences may be bound during the process of replication to its self-encoded proteins, or non-self-encoded cell-derived proteins, nucleic acids or ribonucleoproteins, or complexes between any of these components. In some embodiments of the disclosure, the srRNA (e.g., replicon) is derived from an alphavirus. In some embodiments of the disclosure, an alphavirus srRNA construct (e.g., srRNA, saRNA, or replicon molecule) generally contains the following elements: 5′ viral or defective-interfering RNA sequence(s) required in cis for replication, sequences coding for biologically active alphavirus non-structural proteins (e.g., nsP1, nsP2, nsP3, and nsP4), a subgenomic promoter (sg) for the subgenomic RNA (sgRNA), 3′ viral sequences required in cis for replication, and optionally a polyadenylate tract (poly(A)). In some instances, a subgenomic promoter (sg) that directs expression of a heterologous sequence can be included in the srRNA construct of the disclosure.

Further, the term srRNA molecule (e.g., srRNA, saRNA, or replicon molecule) generally refers to a molecule of positive polarity, or “message” sense, and the srRNA may be of length different from that of any known, naturally-occurring alphavirus. In some embodiments of the present disclosure, the srRNA does not contain at least a portion of the coding sequence for one or more of the alphavirus structural proteins; and/or sequences encoding structural genes can be substituted with heterologous sequences. In those instances, where the srRNA is to be packaged into a recombinant alphavirus particle, it can contain one or more sequences, so-called packaging signals, which serve to initiate interactions with alphavirus structural proteins that lead to particle formation.

The srRNA constructs of the disclosure generally have a length of at least about 2 kb. For example, the srRNA can have a length of at least about 2 kb, at least about 3 kb, at least about 4 kb, at least about 5 kb, at least about 6 kb, at least about 7 kb, at least about 8 kb, at least about 9 kb, at least about 10 kb, at least about 11 kb, at least about 12 kb or more than 12 kb. In some embodiments, the srRNA can have a length of about 4 kb to about 20 kb, about 4 kb to about 18 kb, about 5 kb to about 16 kb, about 6 kb to about 14 kb, about 7 kb to about 12 kb, about 8 kb to about 16 kb, about 9 kb to about 14 kb, about 10 kb to about 18 kb, about 11 kb to about 16 kb, about 5 kb to about 18 kb, about 6 kb to about 20 kb, about 5 kb to about 10 kb, about 5 kb to about 8 kb, about 5 kb to about 7 kb, about 5 kb to about 6 kb, about 6 kb to about 12 kb, about 6 kb to about 11 kb, about 6 Kb to about 10 kb, about 6 Kb to about 9 Kb, about 6 kb to about 8 kb, about 6 kb to about 7 kb, about 7 kb to about 11 kb, about 7 kb to about 10 kb, about 7 kb to about 9 kb, about 7 kb to about 8 kb, about 8 kb to about 11 kb, about 8 kb to about 10 kb, about 8 kb to about 9 kb, about 9 kb to about 11 kb, about 9 kb to about 10 kb, or about 10 kb to about 11 kb. In some embodiments, the srRNA can have a length of about 6 kb to about 14 kb. In some embodiments, the srRNA can have a length of about 6 kb to about 16 kb.

### III. Compositions

### A. Self-Replicating RNAs of the Disclosure

The present disclosure provides, inter alia, functional srRNAs that are de novo assembled from nucleic acid fragments derived from (i) one or more non-functional srRNAs or (ii) one or more non-functional +ssRNA viral genomes. These assembled srRNAs can amplify themselves and initiate expression and/or overexpression of proteins of interest in a host cell or a subject. A srRNA, unlike a mRNA, uses its own encoded polymerase to amplify itself. srRNAs of the present disclosure, such as those based on alphaviruses, generate large amounts of subgenomic mRNAs from which large amounts of proteins of interest can be expressed.

In some embodiments, the functional srRNA can include a heterologous 5′ UTR or a portion thereof. In other embodiments, the functional srRNA can include a heterologous 3′ UTR or a portion thereof. In some embodiments, both 3′ and 5′ UTRs of the functional srRNA can be heterologous. In some embodiments, both 3′ UTR and 5′ UTRs are from the same species, subspecies or strain. In some embodiments, the 3′ UTR and 5′ UTR are from different species, subspecies or strains. The strain can be from a virulent or avirulent version of the virus. In some embodiments, the heterologous 5′ UTR and/or 3′ UTR sequences can be from Chikungunya virus. In some embodiments, the heterologous 5′ UTR and/or 3′ UTR sequences can be from a Chikungunya strain S27. In some embodiments, the heterologous 5′ UTR and/or 3′ UTR sequences can be from a Chikungunya strain DRDE-06.

In some embodiments, the functional srRNA can include one or more of a heterologous nsP, or portions thereof. For example, the heterologous nsP can be nsP1, nsP2, nsP3, or nsP4. In some embodiments the nsP can be from SINV strain AR86 or from SINV strain Girdwood.

In some embodiments, the functional srRNA can include both a heterologous UTR and a heterologous nsP, or portions thereof.

The present disclosure also provides functional srRNAs that are assembled using the methods described herein. The srRNA can include, for example, a 5′ UTR, 26S promoter, nucleic acids for four nonstructural proteins: nsP1, nsP2, nsP3, nsP4, a structural protein and a 3′ UTR. A graphical illustration of the assembly of multiple nucleic acid fragments into a larger construct is shown in FIG. 1B.

In some embodiments, the srRNAs are assembled from non-functional viral genomes or other srRNAs as described in detail below. In some embodiments, the srRNAs contain heterologous nonstructural protein (nsP) or fragments thereof. For example, the heterologous nsP or portion thereof is nsP1, nsP2, nsP3, nsP4, or a portion of any thereof, or a combination of any of the foregoing. As described above, the skilled artisan will understand that a portion of a nucleic acid sequence encoding a nonstructural polypeptide can include enough of the nucleic acid sequence encoding the nonstructural polypeptide to afford putative identification of that polypeptide, either by manual evaluation of the sequence by one skilled in the art, or by computer-automated sequence comparison and identification using algorithms such as BLAST (see, for example, in “Basic Local Alignment Search Tool”; Altschul S F et al., J. Mol. Biol. 215:403-410, 1993). Accordingly, a portion of a nucleotide sequence comprises enough of the sequence to afford specific identification and/or isolation of a nucleic acid fragment comprising the sequence. For example, a portion of a nucleic acid sequence can include at least about 20%, for example, about 30%, about 40%, about 50%, about 60%, about 70%, about 80%, about 90%, about 95% of the full-length nucleic acid sequence.

In some embodiments, the srRNAs contain heterologous UTR or a portion thereof. For example, the heterologous UTR can be a 5′ or a 3′ UTR or a portion of any thereof, or a combination of any of the foregoing.

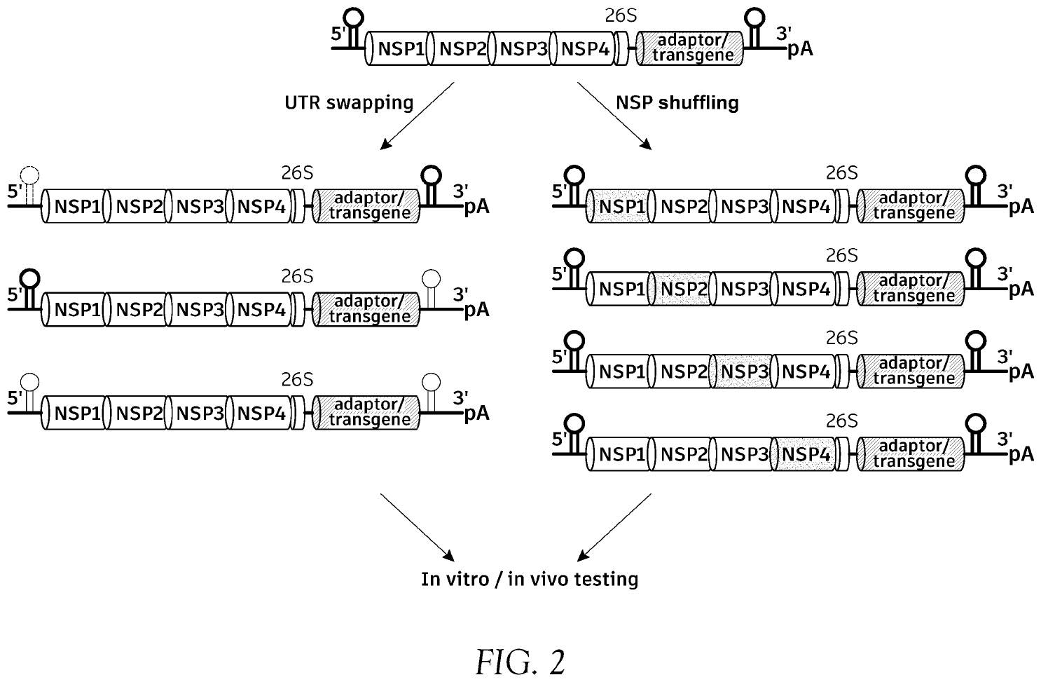

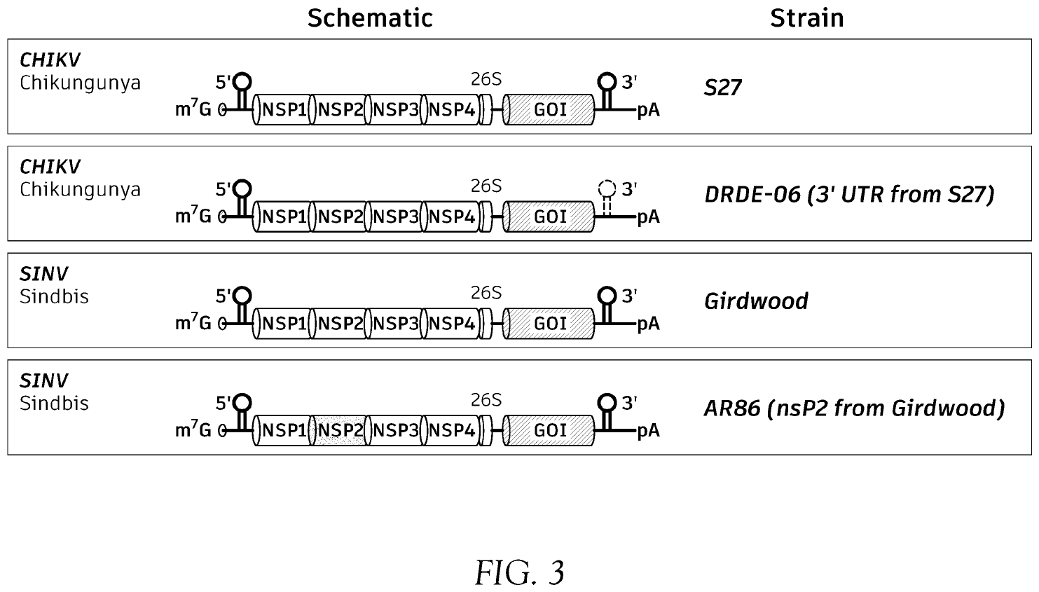

FIG. 2 is a graphical illustration of an exemplary method of assembling a functional srRNAs from a non-functional viral genome. As illustrated in FIG. 2, in some embodiments of the method, swapping one or more UTRs from a different species, subspecies, or strain can be a step for functionalizing a self-replicating RNA molecule. Similarly, in some embodiments of the method, swapping one or more nsPsNSPs or a portion thereof, from a different species, subspecies, or strain can be a step for functionalizing a self-replicating RNA molecule. Exemplary construct designs using the methods described herein are illustrated FIG. 3.

### B. Nucleic Acid Constructs and Vectors

One aspect of the present disclosure relates to nucleic acid constructs comprising or encoding the functional srRNAs described herein. Nucleic acid constructs of the disclosure can be chimeric nucleic acid molecules in which two or more nucleic acid sequences of different origin are assembled into a single nucleic acid molecule. Thus, representative nucleic acid constructs include any constructs that contain (1) nucleic acid sequences, including regulatory and coding sequences that are not found adjoined to one another in nature (e.g., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative nucleic acid constructs can include any recombinant nucleic acid molecules, linear or circular, single-stranded or double-stranded DNA or RNA nucleic acid molecules, derived from any source, such as a plasmid, cosmid, virus, autonomously replicating polynucleotide molecule, phage, capable of genomic integration or autonomous replication, comprising a nucleic acid molecule where one or more nucleic acid sequences have been operably linked. The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The polynucleotide and polypeptide sequences disclosed herein are shown using standard letter abbreviations for nucleotide bases and amino acids as set forth in 37 CFR § 1.82), which incorporates by reference WIPO Standard ST.25 (1998), Appendix 2, Tables 1-6.

Nucleic acid molecules of the present disclosure can be nucleic acid molecules of any length, including nucleic acid molecules that are generally between about 2 kb and 50 kb in length, for example between about 5 kb and about 40 kb, between about 5 kb and about 30 kb, between about 5 kb and about 20 kb, or between about 10 kb and about 50 kb, for example between about 15 kb to 30 kb, between about 20 kb and about 50 kb, between about 20 kb and about 40 kb, between about 5 kb and about 25 kb, or between about 30 kb and about 50 kb. In some embodiments, the nucleic acid molecules are at least 6 kb in length. In some embodiments, the nucleic acid molecules are between about 6 kb and about 20 kb. In some embodiments, the nucleic acid constructs (e.g., vectors or srRNA constructs) of the disclosure generally have a length of at least about 2 kb. For example, the nucleic acid constructs (e.g., vectors or srRNAs) can have a length of at least about 2 kb, at least about 3 kb, at least about 4 kb, at least about 5 kb, at least about 6 kb, at least about 7 kb, at least about 8 kb, at least about 9 kb, at least about 10 kb, at least about 11 kb, at least about 12 kb or more than 12 kb. In some embodiments, the nucleic acid constructs (e.g., vectors or srRNAs) can have a length of about 4 kb to about 20 kb, about 4 kb to about 18 kb, about 5 kb to about 16 kb, about 6 kb to about 14 kb, about 7 kb to about 12 kb, about 8 kb to about 16 kb, about 9 kb to about 14 kb, about 10 kb to about 18 kb, about 11 kb to about 16 kb, about 5 kb to about 18 kb, about 6 kb to about 20 kb, about 5 kb to about 10 kb, about 5 kb to about 8 kb, about 5 kb to about 7 kb, about 5 kb to about 6 kb, about 6 kb to about 12 kb, about 6 kb to about 11 kb, about 6 kb to about 10 kb, about 6 kb to about 9 kb, about 6 kb to about 8 kb, about 6 kb to about 7 kb, about 7 kb to about 11 kb, about 7 kb to about 10 kb, about 7 kb to about 9 kb, about 7 kb to about 8 kb, about 8 kb to about 11 kb, about 8 kb to about 10 kb, about 8 kb to about 9 kb, about 9 kb to about 11 kb, about 9 kb to about 10 kb, or about 10 kb to about 11 kb. In some embodiments, the nucleic acid constructs (e.g., vectors or srRNAs) can have a length of about 6 kb to about 14 kb. In some embodiments, the nucleic acid constructs (e.g., vectors or srRNAs) can have a length of about 6 kb to about 16 kb.

Constructs of the present disclosure can include the necessary elements to direct expression of a nucleic acid sequence of interest that is also contained in the construct. Such elements can include control elements such as a promoter that is operably linked to (so as to direct transcription of) the nucleic acid sequence of interest, and optionally includes a polyadenylation sequence.

One aspect of the present disclosure relates to vectors comprising the nucleic acid construct described above. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon (e.g., srRNA), such as a plasmid, phage, or cosmid, into which another DNA segment may be inserted so as to bring about the replication of the inserted segment. In some embodiments, the expression vector can be an integrating vector. In addition to the components of the construct, the vector can include, for example, one or more selectable markers, one or more origins of replication, such as prokaryotic and eukaryotic origins, at least one multiple cloning site, and/or elements to facilitate stable integration of the construct into the genome of a cell. Two or more constructs can be incorporated within a single nucleic acid molecule, such as a single vector, or can be contained within two or more separate nucleic acid molecules, such as two or more separate vectors.

The molecular techniques and methods by which the nucleic acid constructs of the disclosure can be assembled and characterized are described more fully in the Examples herein of the present application.

In some embodiments, the nucleic acid molecules disclosed herein are produced using recombinant DNA technology (e.g., polymerase chain reaction (PCR) amplification, cloning, etc.) or chemical synthesis. Nucleic acid molecules as disclosed herein include natural nucleic acid molecules and homologs thereof, including, but not limited to, natural allelic variants and modified nucleic acid molecules in which one or more nucleotide residues have been inserted, deleted, and/or substituted, in such a manner that such modifications provide the desired property in effecting a biological activity as described herein.

One skilled in the art will appreciate that nucleic acid molecules, including variants of a naturally-occurring nucleic acid sequence, can be produced using a number of methods known to those skilled in the art (see, for example, Sambrook et al., In: Molecular Cloning, A Laboratory Manual, 2nd Edition, Cold Spring Harbor Press, Cold Spring Harbor, N.Y. (1989)). The sequence of a nucleic acid molecule can be modified with respect to a naturally-occurring sequence from which it is derived using a variety of techniques including, but not limited to, classic mutagenesis techniques and recombinant DNA techniques, such as but not limited to site-directed mutagenesis, chemical treatment of a nucleic acid molecule to induce mutations, restriction enzyme cleavage of a nucleic acid fragment, ligation of nucleic acid fragments, PCR amplification and/or mutagenesis of selected regions of a nucleic acid sequence, recombinational cloning, and chemical synthesis, including chemical synthesis of oligonucleotide mixtures and ligation of mixture groups to “build” a mixture of nucleic acid molecules, and combinations thereof. Nucleic acid molecule homologs can be selected from a mixture of modified nucleic acid molecules by screening for the function of the protein or the replicon, e.g., self-replicating RNA, encoded by the nucleic acid molecule and/or by hybridization with a wild-type gene or fragment thereof, or by PCR using primers having homology to a target or wild-type nucleic acid molecule or sequence.

### C. Pharmaceutical Compositions

The srRNAs, the nucleic acid constructs, vectors, and the recombinant cells of the disclosure can be incorporated into compositions, including pharmaceutical compositions. Such compositions generally include one or more of the nucleic acid constructs, recombinant cells, recombinant polypeptides described and provided herein, and a pharmaceutically acceptable excipient, e.g., carrier. In some embodiments, the compositions of the disclosure are formulated for the prevention, treatment, or management of a health condition such as an immune disease or a microbial infection. For example, the compositions of the disclosure can be formulated as a prophylactic composition, a therapeutic composition, or a pharmaceutical composition comprising a pharmaceutically acceptable excipient, or a mixture thereof. In some embodiments, the compositions of the present disclosure are formulated for use as a vaccine. In some embodiments, the compositions of the present disclosure are formulated for use as an adjuvant.

Accordingly, in one aspect, provided herein are pharmaceutical compositions including a pharmaceutically acceptable excipient and: a) a nucleic acid construct of the disclosure; and/or b) a recombinant cell of the disclosure.

Non-limiting exemplary embodiments of the pharmaceutical compositions of the disclosure can include one or more of the following features. In some embodiments, provided herein are compositions including a nucleic acid construct as disclosed herein and a pharmaceutically acceptable excipient. In some embodiments, provided herein are compositions including a recombinant cell as disclosed herein and a pharmaceutically acceptable excipient.

In some embodiments, the compositions of the disclosure are formulated in a liposome. In some embodiments, the compositions of the disclosure are formulated in a lipid-based nanoparticle (LNP). In some embodiments, the compositions of the disclosure are formulated in a polymer nanoparticle. In some embodiments, the compositions are immunogenic compositions, e.g., composition that can stimulate an immune response in a subject. In some embodiments, the immunogenic compositions are formulated as a vaccine. In some embodiments, the pharmaceutical compositions are formulated as an adjuvant.

In some embodiments, the immunogenic compositions are substantially non-immunogenic to a subject, e.g., compositions that minimally stimulate an immune response in a subject. In some embodiments, the non-immunogenic or minimally immunogenic compositions are formulated as a biotherapeutic. In some embodiments, the pharmaceutical compositions are formulated for one or more of intranasal administration, transdermal administration, intraperitoneal administration, intramuscular administration, intratracheal administration, intranodal administration, intratumoral administration, intraarticular administration, intravenous administration, subcutaneous administration, intravaginal administration, intraocular, rectal, and oral administration.

Pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In these cases, the composition should be sterile and should be fluid to the extent that easy syringability exists. It can be stable under the conditions of manufacture and storage, and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

In some embodiments, the composition is formulated for one or more of intranasal administration, transdermal administration, intraperitoneal administration, intramuscular administration, intratracheal administration, intranodal administration, intratumoral administration, intraarticular administration, intravenous administration, subcutaneous administration, intravaginal administration, intraocular, rectal, and oral administration. In some embodiments, the administered composition results in an increased production of interferon in the subject.

### D. Recombinant Cells

As described in greater detail below, one aspect of the present disclosure relates to recombinant cells that have been engineered to include a nucleic acid construct as described herein, a vector as described herein, and/or include (e.g., express) a srRNA construct as described herein. The present disclosure provides recombinant cells wherein the cells contain a srRNA or a nucleic acid construct encoding a srRNA.

The nucleic acid constructs of the present disclosure can be introduced into a host cell to produce a recombinant cell containing the nucleic acid construct encoding the srRNAs of the present disclosure. Accordingly, prokaryotic or eukaryotic cells that contain a nucleic acid construct encoding a srRNA as described herein are also features of the disclosure. In a related aspect, some embodiments disclosed herein relate to methods of transforming a cell which includes introducing into a host cell, such as an animal cell, a nucleic acid construct as provided herein, and then selecting or screening for a transformed cell. Introduction of the nucleic acid constructs of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, direct micro-injection, nanoparticle-mediated nucleic acid delivery, and the like.

In one aspect, some embodiments of the disclosure relate to recombinant cells, for example, recombinant animal cells that include a nucleic acid construct described herein. The nucleic acid construct can be stably integrated in the host genome, or can be episomally replicating, or present in the recombinant host cell as a mini-circle expression vector for a stable or transient expression. Accordingly, in some embodiments of the disclosure, the nucleic acid construct is maintained and replicated in the recombinant host cell as an episomal unit. In some embodiments, the nucleic acid construct is stably integrated into the genome of the recombinant cell. Stable integration can be completed using classical random genomic recombination techniques or with more precise genome-editing techniques such as using guide RNA directed CRISPR/Cas9 or TALEN genome editing. In some embodiments, the nucleic acid construct present in the recombinant host cell as a mini-circle expression vector for a stable or transient expression.

In some embodiments, the recombinant cell is a prokaryotic cell, such as the bacterium E. coli, or a eukaryotic cell, such as an insect cell (e.g., a mosquito cell or a Sf21 cell), or mammalian cells (e.g., COS cells, NIH 3T3 cells, or HeLa cells). In some embodiments, the cell is in vivo, for example, a recombinant cell in a living body, e.g., cell of a transgenic subject. In some embodiments, the subject is a vertebrate animal or an invertebrate animal. In some embodiments, the subject is an insect. In some embodiments, the subject is a mammalian subject. In some embodiments, the mammalian subject is a human subject. In some embodiments, the cell is ex vivo. In some embodiments, the cell is in vitro. In some embodiments, the recombinant cell is a eukaryotic cell. In some embodiments, the recombinant cell is an animal cell. In some embodiments, the animal cell is a vertebrate animal cell or an invertebrate animal cell. In some embodiments, the recombinant cell is a mammalian cell. In some embodiments, the recombinant cell is selected from the group consisting of a monkey kidney CV1 cell transformed by SV40 (COS-7), a human embryonic kidney cell (e.g., HEK 293 or HEK 293 cell), a baby hamster kidney cell (BHK), a mouse sertoli cell (e.g., TM4 cells), a monkey kidney cell (e.g., CV1), a human cervical carcinoma cell (HeLa), canine kidney cell (e.g., MDCK), buffalo rat liver cell (e.g., BRL 3A), human lung cell (e.g., W138), human liver cell (e.g., Hep G2), mouse mammary tumor (MMT 060562), TRI cell, FS4 cell, a Chinese hamster ovary cell (CHO cell), an African green monkey kidney cell (e.g., Vero cell), a human A549 cell, a human cervix cell, a human CHME5 cell, a human PER.C6 cell, a NS0 murine myeloma cell, a human epidermoid larynx cell, a human fibroblast cell, a human HUH-7 cell, a human MRC-5 cell, a human muscle cell, a human endothelial cell, a human astrocyte cell, a human macrophage cell, a human RAW 264.7 cell, a mouse 3T3 cell, a mouse L929 cell, a mouse connective tissue cell, a mouse muscle cell, and a rabbit kidney cell.

In some embodiments, the recombinant cell is an insect cell, e.g., cell of an insect cell line. In some embodiments, the recombinant cell is a Sf21 cell. Additional suitable insect cell lines include, but are not limited to, cell lines established from insect orders Diptera, Lepidoptera and Hemiptera, and can be derived from different tissue sources. In some embodiments, the recombinant cell is a cell of a lepidopteran insect cell line. In the past few decades, the availability of lepidopteran insect cell lines has increased at about 50 lines per decade. More information regarding available lepidopteran insect cell lines can be found in, e.g., Lynn D. E., Available lepidopteran insect cell lines. Methods Mol Biol. 2007; 388:117-38, which is herein incorporated by reference. In some embodiments, the recombinant cell is a mosquito cell, e.g., a cell of mosquito species within Anopheles (An.), Culex (Cx.) and Aedes (Stegomyia) (Ae.) genera. Exemplary mosquito cell lines suitable for the compositions and methods described herein include cell lines from the following mosquito species: Aedes aegypti, Aedes albopictus, Aedes pseudoscutellaris, Aedes triseriatus, Aedes vexans, Anopheles gambiae, Anopheles stephensi, Anopheles albimanus, Culex quinquefasciatus, Culex theileri, Culex tritaeniorhynchus, Culex bitaeniorhynchus, and Toxorhynchites amboinensis. Suitable mosquito cell lines include, but are not limited to, CCL-125, Aag-2, RML-12, C6/26, C6/36, C7-10, AP-61, A.t. GRIP-1, A.t. GRIP-2, UM-AVE1, Mos.55, Sua1B, 4a-3B, Mos.43, MSQ43, and LSB-AA695BB. In some embodiments, the mosquito cell is a cell of a C6/26 cell line.

### E. Cell Culture

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art

### E. Transgenic Animals

Also provided, in another aspect, are transgenic animals including a nucleic acid construct as described herein, a vector as described herein, and/or include (e.g., express) a srRNA construct as described herein. In some embodiments, the transgenic animal is a vertebrate animal or an invertebrate animal. In some embodiments, the insect is a mosquito. In some embodiments, the transgenic animal is a mammal. In some embodiments, the transgenic mammal is a non-human mammal. Generally, transgenic animals of the present disclosure can be any non-human animal known in the art. Examples of non-human animals suitable for the compositions and methods of the disclosure can include, without limitation, laboratory animals (e.g., mice, rats, hamsters, gerbils, guinea pigs, etc.), livestock (e.g., horses, cattle, pigs, sheep, goats, ducks, geese, chickens, etc.), non-human primates (e.g., apes, chimpanzees, orangutans, monkeys, etc.), fish, amphibians (e.g., frogs, salamanders, etc.), reptiles (e.g., snakes, lizards, etc.), and other animals (e.g., foxes, weasels, rabbits, mink, beavers, ermines, otters, sable, seals, coyotes, chinchillas, deer, muskrats, possums, etc.). The transgenic non-human host animals of the disclosure are prepared using standard methods known in the art for introducing exogenous nucleic acid into the genome of a non-human animal. In some embodiments, the transgenic animal produces a protein of interest.

In some embodiments, the transgenic animal is a vertebrate animal or an invertebrate animal. In some embodiments, the animal is an insect. In some embodiments, the animal is a mammal. In some embodiments, the mammal is a non-human mammal. In some embodiments, the non-human animals of the disclosure are non-human primates. Other animal species suitable for the compositions and methods of the disclosure include animals that are (i) suitable for transgenesis and (ii) capable of rearranging immunoglobulin gene segments to produce an antibody response. Examples of such species include but are not limited to mice, rats, hamsters, rabbits, chickens, goats, pigs, sheep and cows. Approaches and methods for preparing transgenic non-human animals are known in the art. Exemplary methods include pronuclear microinjection, DNA microinjection, lentiviral vector mediated DNA transfer into early embryos and sperm-mediated transgenesis, adenovirus mediated introduction of DNA into animal sperm (e.g., in pig), retroviral vectors (e.g., avian species), somatic cell nuclear transfer (e.g., in goats). The state of the art in the preparation of transgenic domestic farm animals is reviewed in Niemann, H. et al. (2005) Rev. Sci. Tech. 24:285-298.

In some embodiments, the transgenic animal is an insect. In some embodiments, the transgenic animals of the present disclosure are chimeric transgenic animals. In some embodiments, the transgenic animals of the present disclosure are transgenic animals with germ cells and somatic cells containing one or more (e.g., one or more, two or more, three or more, four or more, etc.) nucleic acid constructs of the present disclosure. In some embodiments, the one or more nucleic acid constructs are stably integrated into the genome of the transgenic animals. In some embodiments, the genomes of the transgenic animals of the present disclosure can comprise any of 1, 2, 3, 4, 5, 6, 7, 8, 9, 10, 11, 12, 13, 14, 15 or more copies of the one or more nucleic acid constructs, vectors, and/or srRNAs of the present disclosure.

### IV. Kits

Also provided herein are various kits for the practice of a method described herein. In particular, some embodiments of the disclosure provide kits for producing a polypeptide of interest using the methods described herein. Some other embodiments relate to kits for inducing a pharmacodynamic effect in a subject. Some other embodiments relate to kits for eliciting an immune response in a subject. Some other embodiments relate to kits for the prevention of a health condition in a subject in need thereof. Some other embodiments relate to kits for methods of treating a health condition in a subject in need thereof. Some other embodiments relate to kits for methods of eliciting an immune response in a subject. For example, provided herein, in some embodiments, are kits that include one or more of the srRNAs, nucleic acid constructs, vectors, recombinant cells, and/or pharmaceutical compositions as provided and described herein, as well as written instructions for making and using the same.

In some embodiments, the kits of the disclosure further include one or more means useful for the administration of any one of the provided srRNAs, nucleic acid constructs, vectors, recombinant cells and/or pharmaceutical compositions to a subject. For example, in some embodiments, the kits of the disclosure further include one or more syringes (including pre-filled syringes) and/or catheters (including pre-filled syringes) used to administer any one of the provided nucleic acid constructs, recombinant cells and/or pharmaceutical compositions to a subject. In some embodiments, a kit can have one or more additional therapeutic agents that can be administered simultaneously or sequentially with the other kit components for a desired purpose, e.g., for diagnosing, preventing, or treating a condition in a subject in need thereof.

Any of the above-described kits can further include one or more additional reagents where such additional reagents can be selected from: dilution buffers, reconstitution solutions, wash buffers, control reagents, control expression vectors, negative controls, positive controls, reagents suitable for in vitro production of the provided nucleic acid constructs, recombinant cells and/or pharmaceutical compositions of the disclosure.

In some embodiments, the components of a kit can be in separate containers. In some other embodiments, the components of a kit can be combined in a single container.

In some embodiments, a kit can further include instructions for using the components of the kit to practice the methods disclosed herein. The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

### V. Methods

### Methods for Generating Functional Self-Replicating RNAs

Further provided herein are methods for generating a functional self-replicating RNA (srRNA) from one or more non-functional positive-sense single-stranded viral RNA genomes or non-functional srRNAs. Non-limiting exemplary embodiments of the disclosed methods for generating functional self-replicating RNAs can include one or more of the following features.

In one aspect, provided herein are methods for generating a functional self-replicating RNA (srRNA) including (a) providing a starting material (b) removing one or more RNA polymerase transcription termination sites or cryptic termination sites from the one or more +ssRNA viral genomes or srRNAs; (c) generating a plurality of nucleic acid fragments each comprising a nucleotide sequence derived from the starting material, and (d) assembling the plurality of nucleic acid fragments to generate a de novo functional srRNA assembly.

In another aspect, provided herein are methods for generating a functional self-replicating RNA (srRNA) including (a) providing one or more positive-sense single-stranded RNA (+ssRNA) viral genomes or non-functional srRNAs, wherein at least one of the one or more +ssRNA viral genomes or srRNA is non-functional; (b) removing one or more T7 termination sites or cryptic T7 termination sites from the one or more +ssRNA viral genomes or srRNAs; (c) generating a plurality of nucleic acid fragments each comprising a nucleotide sequence derived from the one or more +ssRNA viral genomes or srRNAs, and (d) assembling the plurality of nucleic acid fragments to generate a de novo functional srRNA assembly.

In another aspect, provided herein are methods for generating a functional self-replicating RNA (srRNA) including (a) providing one or more positive-sense single-stranded RNA (+ssRNA) viral genomes or non-functional srRNAs, wherein at least one of the one or more +ssRNA viral genomes or srRNA is non-functional; (b) removing one or more SP6 termination sites or cryptic SP6 RNA polymerase termination sites from the one or more +ssRNA viral genomes or srRNAs; (c) generating a plurality of nucleic acid fragments each comprising a nucleotide sequence derived from the one or more +ssRNA viral genomes or srRNAs, and (d) assembling the plurality of nucleic acid fragments to generate a de novo functional srRNA assembly.

In some embodiments, the starting material disclosed herein is one or more positive-sense, single-stranded RNA (+ssRNA) viral genomes. In some embodiments, the starting material is only +ssRNA viral genomes. In some embodiments, the starting material is all srRNAs. In some embodiments, the starting material includes a combination of one or more +ssRNA viral genomes and srRNAs, wherein at least one of the one or more +ssRNA viral genomes or srRNA is non-functional. In some embodiments, all of the starting material is non-functional. In some embodiments, the starting material includes a combination of functional and non-functional srRNAs. In some embodiments, the srRNAs and/or +ssRNA viral genomes are full-length. In other embodiments, the srRNAs and/or +ssRNA viral genomes are not full-length but are fragments thereof.

In some embodiments, the starting material includes at least one non-functional srRNA or +ssRNA. In some embodiments, the starting material includes, for example, 1, 2, 3, 4, 5, 6, 7, 8, 9, 10, 11, 12 or more non-functional srRNAs and/or +ssRNA. In other embodiments, the starting material includes from about 1 to about 12, from about 2 to about 11, from about 3 to about 10, from about 4 to about 9, from about 5 to about 8, from about 6 to about 7 non-functional srRNA or +ssRNA.

In some embodiments, the starting material includes various combinations of functional and non-functional srRNAs and/or +ssRNAs or fragments thereof. In some embodiments, 10%, 15%, or 20%, or 25%, or 30%, or 35% or 40%, or 45%, or 50%, or 55%, or 60%, or 65%, or 70%, or 75%, or 80%, or 85%, or 90%, or 95%, 100% of the srRNAs or +ssRNAs are non-functional. In some embodiments, all the srRNAs and/or +ssRNA are non-functional.

Yet, in some embodiments, the starting material includes one or more DNA fragment(s) encoding a desired RNA sequence. In some embodiments, the starting material includes synthetic DNA molecule(s). In some embodiments, the synthetic DNA molecule includes a sequence based on a known srRNA sequence. In some embodiments, the starting material could be a plasmid backbone that includes a nucleic acid encoding a known RNA sequence of interest.

In some embodiments of the methods provided herein, all of the srRNA and/or +ssRNA in the starting material are from the same species. In some embodiments of the methods, all of the srRNA or +ssRNA in the starting material are from different strains or different isolates of the same species. In some embodiments, the srRNA or +ssRNA are derived from different species.

The methods for generating functional srRNAs disclosed herein include the step of removing one or more RNA polymerase transcription termination sites or cryptic termination sites from the starting material (for example, one or more +ssRNA viral genomes or srRNAs). Non-limiting examples of transcription termination sites suitable for the methods disclosed herein include those of bacteriophage RNA polymerases, such as T3, T7, and SP6 DNA-dependent polymerases. In some embodiments, the one or more transcription termination sites include bacteriophage T7 termination sites. In some embodiments, the one or more termination sites include cryptic T7 termination sites. In some embodiments, the termination sites include SP6 termination sites. In some embodiments, the termination sites include SP6 cryptic termination sites.

The skilled artisan will understand that a number of available techniques can be used to remove RNA polymerase transcription termination sites. Such techniques include by way of example, but are not limited to, removal by restriction digestion or by site directed mutagenesis to generate silent mutation(s), or by synthesis of fragment(s) with silent mutation(s) or by subcloning to remove the site(s), etc.

The methods for generating functional srRNAs disclosed herein include the step of generating a plurality of nucleic acid fragments each comprising a nucleotide sequence derived from the one or more +ssRNA viral genomes or srRNAs. The skilled artisan will understand that any technique to generate nucleic acid fragments can be used. For example, PCR amplification, restriction digestion, chemical synthesis, etc.

The methods for generating functional srRNAs disclosed herein include assembling the plurality of nucleic acid fragments to generate a de novo functional srRNA assembly. The skilled artisan will understand that any technique to assemble nucleic acid fragments can be used. For example, by ligation or PCR-based procedures such as by Gibson-assembly techniques, or by fusion PCR, etc. For example, in some embodiments, an initial assembly of a srRNA vector is by restriction digestion of a plasmid backbone, and chemical synthesis of the fragments. In some embodiments, the subsequent reassembly with parts from different strains is by restriction digestion of plasmid, PCR from vectors encoding srRNA vectors, and Gibson assembly.

In some embodiments of the methods provided herein, the plurality of nucleic acid fragments are each about 60 nucleotides to about 5,000 nucleotides in length. In some embodiments, the plurality of nucleic acid fragments are each about 100 to about 4000 nucleotides in length. In other embodiments the plurality of nucleic acid fragments are from about 200 to about 3000 nucleotides in length. In some embodiments, the nucleic acid fragments disclosed herein are from about 200 to about 1000 nucleotides in length. In other embodiments, the nucleic acid fragments are from about 200 to about 500 nucleotides in length. In some embodiments, the plurality of nucleic acid fragments are single-stranded or double-stranded nucleic acids.

In some embodiments of the methods provided herein the de novo functional srRNA assembly can be devoid of the nucleic acid sequence encoding one or more viral structural proteins. In some aspects, the de novo functional srRNA assembly can be devoid of a substantial portion of the nucleic acid sequence encoding one or more viral structural proteins. The skilled artisan will understand that a substantial portion of a nucleic acid sequence encoding a viral structural polypeptide can include enough of the nucleic acid sequence encoding the viral structural polypeptide to afford putative identification of that polypeptide, either by manual evaluation of the sequence by one skilled in the art, or by computer-automated sequence comparison and identification using algorithms such as BLAST (see, for example, in “Basic Local Alignment Search Tool”; Altschul S F et al., J. Mol. Biol. 215:403-410, 1993). Accordingly, a substantial portion of a nucleotide sequence comprises enough of the sequence to afford specific identification and/or isolation of a nucleic acid fragment comprising the sequence. For example, a substantial portion of a nucleic acid sequence can include at least about 20%, for example, about 30%, about 40%, about 50%, about 60%, about 70%, about 80%, about 90%, about 95% of the full-length nucleic acid sequence. In some embodiments, the alphavirus srRNA vector is devoid of the entire sequence encoding viral structural proteins, e.g., the de novo functional srRNA assembly comprises no nucleic acid sequence encoding viral structural proteins.

In some embodiments of the methods provided herein, the +ssRNA viral genomes or srRNAs can be from a virus belonging to the Alphavirus genus of the Togaviridae family. In some embodiments, the +ssRNA viral genome or srRNAs is of an alphavirus species belonging to VEEV/EEEV group, or the SFV group, or the SINV group. In some embodiments, the +ssRNA viral genomes or srRNAs can be that of an alphavirus such as the Eastern equine encephalitis virus (EEEV), Venezuelan equine encephalitis virus (VEEV), Everglades virus (EVEV), Mucambo virus (MUCV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu virus (NDUV), Madariaga virus (MADV), and Buggy Creek virus. In some embodiments, the +ssRNA viral genome or srRNAs can be that of Eastern equine encephalitis virus (EEEV), Chikungunya virus (CHIKV), Sindbis virus (SINV), Venezuelan equine encephalitis virus (VEE), Madariaga virus (MADV), Western equine encephalitis virus (WEEV), or Semliki Forest virus (SFV). In some embodiments, the alphavirus is VEEV. In some embodiments, the alphavirus is EEEV. In some embodiments, the alphavirus is CHIKV. In some embodiments, the alphavirus is SINV.

Suitable wild-type alphavirus sequences are well-known and are available from sequence depositories, such as NCBI Genbank, the American Type Culture Collection, Rockville, Md. Representative examples of suitable alphaviruses include Aura (ATCC VR-368), Bebaru virus (ATCC VR-600, ATCC VR-1240), Cabassou (ATCC VR-922), Chikungunya virus (ATCC VR-64, ATCC VR-1241), Eastern equine encephalomyelitis virus (ATCC VR-65, ATCC VR-1242), Fort Morgan (ATCC VR-924), Getah virus (ATCC VR-369, ATCC VR-1243), Kyzylagach (ATCC VR-927), Mayaro (ATCC VR-66), Mayaro virus (ATCC VR-1277), Middleburg (ATCC VR-370), Mucambo virus (ATCC VR-580, ATCC VR-1244), Ndumu (ATCC VR-371), Pixuna virus (ATCC VR-372, ATCC VR-1245), Ross River virus (ATCC VR-373, ATCC VR-1246), Semliki Forest (ATCC VR-67, ATCC VR-1247), Sindbis virus (ATCC VR-68, ATCC VR-1248), Tonate (ATCC VR-925), Triniti (ATCC VR-469), Una (ATCC VR-374), Venezuelan equine encephalomyelitis (ATCC VR-69, ATCC VR-923, ATCC VR-1250 ATCC VR-1249, ATCC VR-532), Western equine encephalomyelitis (ATCC VR-70, ATCC VR-1251, ATCC VR-622, ATCC VR-1252), Whataroa (ATCC VR-926), and Y-62-33 (ATCC VR-375).

In some embodiments of the methods of the present disclosure, the method further comprises incorporating a nucleic acid sequence encoding a heterologous gene into the de novo srRNA assembly. The heterologous gene can be any gene of interest (GOI).

The polypeptide encoded by a GOI can generally be any polypeptide, and can be, for example a therapeutic polypeptide, a prophylactic polypeptide, a diagnostic polypeptide, a nutraceutical polypeptide, an industrial enzyme, and a reporter polypeptide. In some embodiments, the GOI encodes a polypeptide selected from the group consisting of an antibody, an antigen, an immune modulator, an enzyme, a signaling protein, and a cytokine. In some embodiments, the coding sequence of the GOI is optimized for expression at a level higher than the expression level of a reference coding sequence. In some embodiments, the coding sequence of the GOI is optimized for enhanced RNA stability. Several methodologies and techniques useful for evaluation of RNA stability are known, including various in silico methodologies and/or empirical stress-testing of storage of srRNAs with different GOI codon usage, and its effects on srRNA potency (e.g., examine dsRNA in cells following transfection) and gene expression. Additional information in this regard can be found in, for example, Wayment-Steele, H. et al. (2021). Cold Spring Harbor Laboratory (doi.org/10.1101/2020.08.22.262931). In some embodiments, the polypeptide encoded by the GOI is a recombinant polypeptide.

In some embodiments, the polypeptide encoded can be a sequence that results in multiple polypeptide products, linked by self-cleaving peptides (e.g. P2A), or separated by IRES sequence(s).

In some embodiments of the methods provided herein, at least a portion of the nucleic acid sequence encoding the viral structural proteins of the de novo srRNA assembly is replaced by an expression cassette comprising a heterologous gene operably linked to a promoter. In some embodiments, the heterologous gene can be operably linked to a subgenomic (sg) promoter. The sg promoter can be a 26S subgenomic promoter.

In other embodiments of the methods of the present disclosure, the method further comprises removing one or more restriction enzyme sites from one or more of the non-functional +ssRNA genome or srRNAs. In some embodiments, at least one of the removed restriction enzyme sites is recognized by a restriction enzyme suitable for linearization of the de novo srRNA assembly or for insertion of the heterologous gene into the de novo srRNA assembly.

In some embodiments of the methods provided herein, the generated functional srRNA assembly comprises a 3′ polyadenylate tract (poly(A) tail). In some embodiments, the 3′ poly(A) tail comprises at least 11 adenine nucleotides. In some embodiments, the 3′ poly(A) tail comprises at least 15 nucleotides. Yet in some embodiments, the 3′ poly(A) tail comprises at least 20 nucleotides, at least 25 nucleotides, at least 30 nucleotides, at least 35 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides or at least 250 nucleotides. In some embodiments, the 3′ poly(A) tail is from about 11 to about 300, or from about 20 to about 250, or from about 30 to about 200, or from about 40 to about 150, or from about 50 to about 100 adenine nucleotides long.

In some embodiments, the methods provided herein further comprise replacing one or more untranslated regions (UTR) or a portion thereof in the de novo srRNA assembly with a UTR from a different species or subspecies or strain of the alphavirus or the +ssRNA viral genome. In some embodiments, only the 5′ UTR or a portion thereof is replaced. In some embodiments, only the 3′ UTR or a portion thereof is replaced. In some embodiments, one or more of the UTRs are replaced by a heterologous UTR from a different +ssRNA virus subspecies or different +ssRNA virus species, or a different +ssRNA virus strain relative to the species of the non-functional viral genome. In some embodiments, both 3′ UTR and 5′ UTR are replaced. In such embodiments, both 3′ UTR and 5′ UTR are replaced by heterologous UTRs from the same +ssRNA virus species, subspecies or strain. In some embodiments, the 3′ UTR and 5′ UTR are replaced by heterologous UTRs from different +ssRNA virus species, subspecies or strain. The strain can be from a virulent or avirulent version of the virus. In some embodiments, the heterologous 5′ UTR and/or 3′ UTR sequences can be from Chikungunya virus. In some embodiments, the heterologous 5′ UTR and/or 3′ UTR sequences can be from a Chikungunya strain S27. In some embodiments, the heterologous 5′ UTR and/or 3′ UTR sequences can be from a Chikungunya strain DRDE-06.

In some embodiments of the methods of the present disclosure, the method includes replacing a non-structural protein (nsP), or a portion thereof, in the de novo srRNA assembly with a heterologous nsP. The heterologous nsP or portion thereof can be from another +ssRNA virus species or subspecies. In some aspects, the nsP or portion thereof is derived from another strain of the same +ssRNA virus species. In some embodiments, the nsP or portion thereof is nsP1, nsP2, nsP3, nsP4, or a portion of any thereof.

During viral replication, each of the nsP subunits (e.g., nsP1, nsP2, nsP3, nsP4) of an nsP polyprotein complex is processed separately into an individual protein. These proteins then come together to form an nsP polyprotein complex that performs genomic and subgenomic transcription function. Without being bound to any particular theory, it is hypothesized that each nsP itself is reasonably biologically self-contained in terms of contributing to the overall function of the srRNA and should be treated as a discrete modular unit. In some embodiments, of the method of functionalizing a non-functional alphavirus genome or srRNA, each of the nsP subunits is treated as a discrete modular unit and can be replaced (swapped) by a corresponding modular unit from another virus (e.g., another species or another strain of the same species), resulting in a chimeric virus with new characteristics. The presently disclosed method allows for a scientific assessment of the effect of swapping out nsPs in a following minimal set: (1) a srRNA that does not undergo successful replication and/or protein expression in cells in vitro; and (2) a srRNA that does undergo successful replication and/or protein expression in cells in vitro. This approach rapidly gives information on which nsP is creating issues for any given strain without the need to create a large number of new constructs. In some embodiments of the methods provided herein, the method includes selecting the nsP or UTR from versions of a virulent species of a +ssRNA virus. In some embodiments of the methods provided herein, the method includes selecting the nsP or UTR from versions of an avirulent species of a +ssRNA virus. In some embodiments, the selection of species or subspecies of the +ssRNA can be triaged by selecting virulent or avirulent versions based on whether immune-modulatory mechanisms that have been characterized in the NSP regions or UTR regions. A person of skill in the art would readily appreciate that virulent species or subspecies or strains frequently have more or less immunostimulatory-inducing or inflammation-inducing activities relative to avirulent species or subspecies or strains. This can have benefits or determents to the utility of a given vector for its application depending on whether enhanced immunogenicity is desired (vaccine) or suppressed immunogenicity is desired (biotherapeutic GOI). If the virulence phenotype is linked to a sequence in the nsP or UTR, it is likely to have a profound effect on the ultimate application of the functionalized srRNA vector. For example, when functionalizing an avirulent strain into an srRNA vector, it may be desirable to select nsP(s) and/or UTR(s) from other avirulent species or subspecies or strains, or it may be desirable to select nsP(s) and/or UTR(s) from virulent species or subspecies or strains. In some embodiments, when functionalizing a virulent strain into an srRNA vector, it may be desirable to select nsP(s) and/or UTR(s) from avirulent species or subspecies or strains. In some embodiments, it may be desirable to select nsP(s) and/or UTR(s) from other virulent species or subspecies or strains.

In some embodiments, the methods provided herein include assessing the functionality of the de novo srRNA assembly. In some embodiments, assessing the functionality is carried out in vitro. In some embodiments, assessing the functionality is carried out in vivo. In some embodiments, assessing the functionality is carried out ex vivo. In other embodiments, the assessing functionality includes analyzing one or more of the de novo srRNA assemblies constructs for capability of self-replicating in vivo. In other embodiments, the assessing functionality includes analyzing one or more of the de novo srRNA assemblies constructs for capability of self-replicating ex vivo.

In some embodiments of the methods provided herein, assessing the functionality of the alphavirus genome or srRNA is performed using an assay including: detection of RNA replication, detection of viral protein expression, detection of cytopathic effect (CPE), and detection of heterologous gene expression.

In some embodiments, assessing the functionality of the de novo srRNA assembly is performed by incorporating a nucleic acid sequence encoding a heterologous gene into the de novo srRNA assembly. In other embodiments, the methods comprise assessing functionality of the assembled constructs without incorporating a nucleic acid sequence encoding a heterologous gene into one or more of the de novo srRNA assemblies. In some embodiments, the non-functionality of the alphavirus genome or srRNA is determined by a deficiency in self-replication within a host cell.

In general, the functionality of the srRNAs can be evaluated by using one or more assays and methodologies known in the art. Examples of suitable analytical techniques for assessing the functionality include, but are not limited to, immunoblotting analysis, fluorescence flow cytometry analysis, enzyme-linked immunoassay analysis, immunogenicity analysis, bioactivity analysis, and efficacy in a disease model. In some embodiments, the non-functionality of the assembled srRNA is determined by a deficiency in self-replication within a host cell. In particular, a non-functional srRNA or viral genome can be identified as being incapable of self-replication within a cell culture or primary cell line, for example, but not limited to, BHK, VERO, or HEK293. An exemplary assay that can be used for detection of replicating vectors includes an in vitro potency assay. In this assay, replicating vectors are detected using an assay that measures replication efficiency by capturing intermediate dsRNA and comparable protein expression in individual cells by an antigen-specific monoclonal antibody, J2. Test srRNA is diluted and directly electroporated into cells. When the test srRNA (input srRNA composition) is encapsulated or adsorbed to a non-viral delivery system, a detergent (or other extraction method) is used to extract the srRNA from the delivery system before it is electroporated into cells. After sufficient incubation, the cells are fixed and immunoassayed with a fluorophore-conjugated antibody (J2) that specifically detects the dsRNA replication intermediate of the vector. Signal-positive cells indicate the presence of an intact and functional srRNA, which can be quantified by fluorescence flow cytometry. The assay readout is the frequency of positive cells per ng of RNA transfected. There is a dose-responsive frequency of dsRNA+transfected cells which results in the ability to generate a sigmoidal curve that is similar to the standard curve generated by the widely used enzyme-linked immunosorbent assay (ELISA) for quantification of other biological molecules. A srRNA reference standard can be used to help mitigate cell-based assay variability and enable comparison of potency across assays. A srRNA standard can be an aliquot of a large prep that is stored at −80° C. and the potency of each test RNA is defined relative to the standard.

In some embodiments, a similar strategy for quantification of virus replicon particle (VRPs) titers can be employed by infection of cells with serial dilutions of the particles followed by immunoassay with the J2 antibody.

In some embodiments, the functionality of an srRNA is assessed by measuring proteins expressed from the srRNA. For example, RNA can be transformed by electroporation into BHK-21 or Vero cells (e.g. 4D-Nucleofector™, Lonza). After sufficient incubation following transformation, the cells are fixed and permeabilized (eBioscience™ Foxp3/Transcription Factor Staining Buffer Set, Invitrogen) and stained using a fluorophpore-conjugated monoclonal antibody specific to a protein of interest to quantify the frequency of protein+ cells and the mean fluorescence intensity (MFI) of the protein in individual cells by fluorescence flow cytometry.

An exemplary workflow of the present disclosure begins with providing one or more non-functional genomes or srRNAs or a combination of functional and non-functional srRNAs and removing any RNA polymerase termination site or cryptic termination site such as a T7 termination site, a cryptic T7 termination site, an SP6 termination site, or an SP6 cryptic termination site from one or more of the srRNAs thereby generating a plurality of nucleic acid fragments, de novo assembling from about 60 bp to 5,000 bp fragments to result in a template DNA that can be used for in vitro transcription to generate srRNAwith a poly(A) tail having at least 11 adenine nucleotides. The assembled srRNA can include a promoter, a 5′ UTR, and sequences encoding: nsP1, nsP2, nsP3 and nsP4, 26S subgenomic promoter, an adaptor and/or a transgene, a 3′ UTR and a poly(A), followed by a terminator or restriction site. The functionality of the de novo assembled srRNA can be tested in vivo or in vitro using no heterologous gene or a heterologous gene, or both. If the srRNA is found to be non-functional, e.g., no replication or protein expression occurred, one or more of the nsPs or portions thereof are swapped or replaced with a heterologous nsP. Alternatively if the assembled srRNA is found non-functional one or more UTRs can be replaced by heterologous UTRs. The UTRs as described above, can be 5′ or 3′ UTRs or both. In some embodiments, one or more of the srRNA fragments that are used to generate the functional srRNAs of the present disclosure are synthesized de novo.

Another exemplary workflow for functionalizing a srRNA vector assembly is as follows. Several nucleic acid fragments (can be 100 bp-12 Kb) can be synthesized (by any known method) based on a reference alphavirus +ssRNA genome sequence or srRNA from a gene repository such as Genbank (starting material; for example strain 1 of a +ss RNA virus). The fragments can be synthesized with a unique restriction enzyme cut site in place of the coding sequence of the viral structural genes. An RNA polymerase promoter is included upstream of the genome sequence, and a polyA sequence can be downstream, if not already present, followed by a unique restriction enzyme site that is followed by a terminator sequence followed by another unique restriction enzyme cut site. The parts can be combined by any known method such as, by way of example, in a five-piece Gibson Assembly® reaction (e.g., a restriction-digest linearized plasmid backbone and the four synthesized fragments). The above vector is surprisingly non-functional (i.e., it does not undergo self-replication and/or is not capable of expression of a tested transgene/protein). The above vector can be functionalized by replacing one or more endogenous nsPs or UTRs by a heterologous equivalent. For example, by replacing the endogenous nsP2 with a heterologous nsP2 from a srRNA of a 2nd strain of virus 1 as follows. The above vector is linearized by restriction digestion, and the large fragment is isolated by gel extraction following gel electrophoresis. This linearized product may lack part of the other endogenous nsPs. The missing portion of the endogenous nsPs is generated by PCR from the non-functional srRNA vector template described above. The heterologous nsP2 from strain 2 is generated by PCR from a different srRNA vector template (that is prepared in a similar manner to the strain 1 srRNA vector above) based on the second strain published reference sequence. The parts are combined in a Gibson Assembly® reaction to result in a functional srRNA vector. In a second example, the endogenous 3′ UTR is replaced with a heterologous 3′ UTR from a srRNA of a 2nd strain of virus 1 as follows. The non-functional vector is linearized by restriction digestion, and the large fragment is isolated by gel extraction following gel electrophoresis. The linearized product may lack part of the other features of the srRNA vector such as the polyA, restriction site(s), or T7 terminator. The heterologous 3′ UTR and missing vector features are generated by PCR from a different srRNA vector template (that is prepared in a similar manner to the strain 1 srRNA vector above) based on the second strain published reference sequence. The parts are combined in a Gibson Assembly® reaction to result in a functional srRNA vector.

The functionality of the srRNA generated according to the methods of the disclosure can be assayed using an in vitro transcription procedure, for instance as described in Example 4 below.

The methods for generating functional srRNAs disclosed herein can be varied, as appreciated by a person of skill in the art, but still remain within the scope of the invention. Given a non-functional srRNA, a functional srRNA can be generated by assembling nucleic acid fragments encoding all or parts of the non-functional srRNA after removal of any RNA polymerase termination sites or cryptic termination sites.

Also in accordance with the methods for generating functional srRNA of the present disclosure, a functional srRNA can be generated by assembling nucleic acid fragments encoding all or parts of the non-functional srRNA after removal of any RNA polymerase termination sites or cryptic termination sites (such as T7 termination sites, cryptic T7 termination sites, SP6 termination sites, or cryptic SP6 termination sites) and using a fragment encoding a heterologous UTR as described herein.

In some embodiments of the methods for generating functional srRNA of the present disclosure, a functional srRNA can be generated by assembling nucleic acid fragments encoding all or parts of the non-functional srRNA after removal of any RNA polymerase termination sites or cryptic termination sites (such as T7 termination sites, cryptic T7 termination sites, SP6 termination sites, or cryptic SP6 termination sites) and using one or more fragments encoding a heterologous nsP as described above.

### Methods of Inducing a Pharmacodynamic Effect, Preventing, or Treating Health Conditions

In an aspect, provided herein are methods for inducing a pharmacodynamic effect in a subject, the methods include administering to the subject a composition including: (a) a functional srRNA as disclosed herein, (b) a nucleic acid construct as disclosed herein, (c) a recombinant cell as disclosed herein, and/or (d) a pharmaceutical composition as disclosed herein. In some embodiments, the pharmacodynamic effect includes eliciting an immune response in the subject.

Examples of pharmacodynamic effects that can be analyzed include: immunogenicity effect (e.g., eliciting an immune response in vivo), a biomarker response, a therapeutic effect, a prophylactic effect, a desired effect, an undesired effect, an adverse effect, and effect in a disease model. In some embodiments, the assessment of pharmacodynamic effects includes assessing induction of an immune response in vivo. In some embodiments, the assessment of pharmacodynamic effects includes assessing induction of cytokine pathways that can potentiate an immune response and prevent angiogenesis and metastasis.

In another aspect, provided herein are methods for preventing or treating a health condition in a subject, the method includes administering to the subject a composition including: (a) a functional srRNA as disclosed herein, (b) a nucleic acid construct as disclosed herein, (c) a recombinant cell as disclosed herein, and/or (d) a pharmaceutical composition as disclosed herein. In some embodiments, the pharmacodynamic effect includes eliciting an immune response in the subject.

Administration of any one of the pharmaceutical or therapeutic compositions described herein, e.g., nucleic acid constructs, recombinant cells, can be used in the treatment of relevant health conditions, such as proliferative disorders (e.g., cancers) and chronic infections (e.g., viral infections). In some embodiments, the nucleic acid constructs, recombinant cells, and/or pharmaceutical compositions as described herein can be incorporated into therapeutic agents for use in methods of treating an individual or subject who has, who is suspected of having, or who may be at high risk for developing one or more relevant health conditions or diseases. Exemplary health conditions or diseases can include, without limitation, cancers, immune diseases, autoimmune diseases, inflammatory diseases, gene therapy, gene replacement, cardiovascular diseases, age-related pathologies, acute infection, and chronic infection. In some embodiments, the individual is a patient under the care of a physician.

Examples of autoimmune diseases suitable for the methods of the disclosure include, but are not limited to, rheumatoid arthritis, osteoarthritis, Still's disease, Familiar Mediterranean Fever, systemic sclerosis, multiple sclerosis, ankylosing spondylitis, Hashimoto's tyroidism, thyroiditis, systemic lupus erythematosus, Sjögren's syndrome, diabetic retinopathy, diabetic vasculopathy, diabetic neuralgia, insulitis, psoriasis, alopecia areata, warm and cold autoimmune hemolytic anemia (AIHA), pernicious anemia, acute inflammatory diseases, autoimmune adrenalitis, chronic inflammatory demyelinating polyneuropathy (CIDP), Lambert-Eaton syndrome, lichen sclerosis, Lyme disease, Graves disease, Behçet's disease, Ménière's disease, reactive arthritis (Reiter's syndrome), Churg-Strauss syndrome, Cogan syndrome, CREST syndrome, pemphigus vulgaris and pemphigus foliaceus, bullous pemphigoid, polymyalgia rheumatica, polymyositis, primary biliary cirrhosis, pancreatitis, peritonitis, psoriatic arthritis, rheumatic fever, sarcoidosis, scleroderma, celiac disease, stiff-man syndrome, Takayasu arteritis, transient gluten intolerance, autoimmune uveitis, vitiligo, polychondritis, dermatitis herpetiformis (DH) or Duhring's disease, fibromyalgia, Goodpasture syndrome, Guillain-Barré syndrome, Hashimoto thyroiditis, autoimmune hepatitis, inflammatory bowel disease (IBD), Crohn's disease, ulcerative colitis, myasthenia gravis, immune complex disorders, glomerulonephritis, polyarteritis nodosa, anti-phospholipid syndrome, polyglandular autoimmune syndrome, idiopathic pulmonary fibrosis, idiopathic thrombocytopenia purpura (ITP), urticaria, autoimmune infertility, juvenile rheumatoid arthritis, sarcoidosis, and autoimmune cardiomyopathy.

Non-limiting examples of infection suitable for the methods of the disclosure include infections with viruses such as human immunodeficiency virus (HIV), hepatitis B virus (HBV), hepatitis B virus (HCV), Cytomegalovirus (CMV), respiratory syncytial virus (RSV), human papillomavirus (HPV), Epstein-Barr virus (EBV), severe acute respiratory syndrome coronavirus 2 (SARS-CoV2), severe acute respiratory syndrome coronavirus (SARS-CoV), Middle East Respiratory Syndrome (MERS), influenza virus, and Ebola virus. Additional infections suitable for the methods of the disclosure include infections with intracellular parasites such as Leishmania, Rickettsia, Chlamydia, Coxiella, Plasmodium, Brucella, mycobacteria, Listeria, Toxoplasma and Trypanosoma. In some embodiments, the srRNA constructs, nucleic acid constructs, recombinant cells, and/or pharmaceutical compositions, can be useful in the treatment and/or prevention of immune diseases, autoimmune diseases, or inflammatory diseases such as, for example, glomerulonephritis, inflammatory bowel disease, nephritis, peritonitis, psoriatic arthritis, osteoarthritis, Still's disease, Familiar Mediterranean Fever, systemic scleroderma and sclerosis, inflammatory bowel disease (IBD), Crohn's disease, ulcerative colitis, acute lung injury, meningitis, encephalitis, uveitis, multiple myeloma, glomerulonephritis, nephritis, asthma, atherosclerosis, leukocyte adhesion deficiency, multiple sclerosis, Raynaud's syndrome, Sjögren's syndrome, juvenile onset diabetes, Reiter's disease, Behçet's disease, immune complex nephritis, IgA nephropathy, IgM polyneuropathies, immune-mediated thrombocytopenias, hemolytic anemia, myasthenia gravis, lupus nephritis, lupus erythematosus, rheumatoid arthritis (RA), ankylosing spondylitis, pemphigus, Graves' disease, Hashimoto's thyroiditis, small vessel vasculitides, Omen's syndrome, chronic renal failure, autoimmune thyroid disease, acute infectious mononucleosis, HIV, herpes virus associated diseases, human virus infections, coronavirus, other enterovirus, herpes virus, influenza virus, parainfluenza virus, respiratory syncytial virus or adenovirus infection, bacteria pneumonia, wounds, sepsis, cerebral stroke/cerebral edema, ischemia-reperfusion injury, and hepatitis C.

Non-limiting examples of inflammatory suitable for the methods of the disclosure include inflammatory diseases such as asthma, inflammatory bowel disease (IBD), chronic colitis, splenomegaly, and rheumatoid arthritis.

In some embodiments, the subject is vertebrate animal or an invertebrate animal. In some embodiments, the subject is a mammalian subject. In some embodiments, the mammalian subject is a human subject.

Accordingly, in one aspect, provided herein are methods for eliciting an immune response in a subject in need thereof, the method includes administering to the subject a composition including: a) a nucleic acid construct of the disclosure; b) a recombinant cell of the disclosure; and/or c) a pharmaceutical composition of the disclosure.

In another aspect, provided herein are methods for preventing and/or treating a health condition in a subject in need thereof, the method includes prophylactically or therapeutically administering to the subject a composition including: a) a nucleic acid construct of the disclosure; b) a recombinant cell of the disclosure; and/or c) a pharmaceutical composition of any one of the disclosure.

In some embodiments, the health condition is a proliferative disorder or a microbial infection. In some embodiments, the subject has or is suspected of having a condition associated with proliferative disorder or a microbial infection.

In some embodiments, the disclosed composition is formulated to be compatible with its intended route of administration. For example, the nucleic acid constructs, recombinant cells, and/or pharmaceutical compositions of the disclosure can be given orally or by inhalation, but it is more likely that they will be administered through a parenteral route. Examples of parenteral routes of administration include, for example, intravenous, intranodal, intradermal, subcutaneous, transdermal (topical), transmucosal, intravaginal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Dosage, toxicity and therapeutic efficacy of such subject nucleic acid constructs, recombinant cells, and/or pharmaceutical compositions of the disclosure can be determined by standard pharmaceutical procedures in cell cultures or experimental animals, e.g., for determining the LD50 (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are generally suitable. While compounds that exhibit toxic side effects can be used, care should be taken to design a delivery system that targets such compounds to the site of affected tissue in order to minimize potential damage to uninfected cells and, thereby, reduce side effects.

For example, the data obtained from the cell culture assays and animal studies can be used in formulating a range of dosage for use in humans. The dosage of such compounds lies generally within a range of circulating concentrations that include the ED₅₀ with little or no toxicity. The dosage can vary within this range depending upon the dosage form employed and the route of administration utilized. For any compound used in the method of the disclosure, the therapeutically effective dose can be estimated initially from cell culture assays. A dose can be formulated in animal models to achieve a circulating plasma concentration range that includes the IC₅₀ (e.g., the concentration of the test compound which achieves a half-maximal inhibition of symptoms) as determined in cell culture. Such information can be used to more accurately determine useful doses in humans. Levels in plasma can be measured, for example, by high performance liquid chromatography.

The therapeutic compositions described herein, e.g., nucleic acid constructs, recombinant cells, and/or pharmaceutical compositions, can be administered one from one or more times per day to one or more times per week; including once every other day. The skilled artisan will appreciate that certain factors can influence the dosage and timing required to effectively treat a subject, including but not limited to the severity of the disease, previous treatments, the general health and/or age of the subject, and other diseases present. Moreover, treatment of a subject with a therapeutically effective amount of the subject multivalent polypeptides and multivalent antibodies of the disclosure can include a single treatment or, can include a series of treatments. In some embodiments, the compositions are administered every 8 hours for five days, followed by a rest period of 2 to 14 days, e.g., 9 days, followed by an additional five days of administration every 8 hours. With regard to nucleic acid constructs the therapeutically effective amount of a nucleic acid construct (e.g., an effective dosage) depends on the nucleic acid construct selected. For instance, single dose amounts in the range of approximately 0.001 to 0.1 mg/kg of patient body weight can be administered. In some embodiments, about 0.005, 0.01, 0.05 mg/kg can be administered. In some embodiments, single dose amounts in the range of approximately 0.03 μg to 300 μg/kg of patient body weight can be administered. In some embodiments, single dose amounts in the range of approximately 0.3 mg to 3 mg/kg of patient body weight can be administered.

As discussed supra, a therapeutically effective amount includes an amount of a therapeutic composition that is sufficient to promote a particular effect when administered to a subject, such as one who has, is suspected of having, or is at risk for a health condition, e.g., a disease or infection. In some embodiments, an effective amount includes an amount sufficient to prevent or delay the development of a symptom of the disease or infection, alter the course of a symptom of the disease or infection (for example but not limited to, slow the progression of a symptom of the disease or infection), or reverse a symptom of the disease or infection. It is understood that for any given case, an appropriate effective amount can be determined by one of ordinary skill in the art using routine experimentation.

The efficacy of a treatment including a disclosed therapeutic composition for the treatment of disease or infection can be determined by the skilled clinician. However, a treatment is considered effective treatment if at least any one or all of the signs or symptoms of disease or infection are improved or ameliorated. Efficacy can also be measured by failure of an individual to worsen as assessed by hospitalization or need for medical interventions (e.g., progression of the disease or infection is halted or at least slowed). Methods of measuring these indicators are known to those of skill in the art and/or described herein. Treatment includes any treatment of a disease or infection in a subject or an animal (some non-limiting examples include a human, or a mammal) and includes: (1) inhibiting the disease or infection, e.g., arresting, or slowing the progression of symptoms; or (2) relieving the disease or infection, e.g., causing regression of symptoms; and (3) preventing or reducing the likelihood of the development of symptoms.

In some embodiments, the nucleic acid constructs, recombinant cells and/or pharmaceutical compositions of the disclosure can be administered to a subject in a composition having a pharmaceutically acceptable carrier and in an amount effective to stimulate an immune response. Generally, a subject can be immunized through an initial series of injections (or administration through one of the other routes described below) and subsequently given boosters to increase the protection afforded by the original series of administrations. The initial series of injections and the subsequent boosters are administered in such doses and over such a period of time as is necessary to stimulate an immune response in a subject. In some embodiments, the administered composition results in an increased production of interferon in the subject. In some embodiments of the disclosed methods, the subject is a mammal. In some embodiments, the mammal is human.

As described above, pharmaceutically acceptable carriers suitable for injectable use include sterile aqueous solutions (where water soluble) or dispersions and sterile powders for the extemporaneous preparation of sterile injectable solutions or dispersions. In these cases, the composition can be sterile and can be fluid to the extent that easy syringability exists. The composition can be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, etc.), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, asorbic acid, thimerosal, and the like.

Sterile injectable solutions can be prepared by incorporating the nucleic acid constructs and recombinant cells in the required amount in an appropriate solvent with one or a combination of ingredients enumerated above, as required, followed by filtered sterilization.

When the nucleic acid constructs, recombinant cells, and/or pharmaceutical compositions are suitably protected, as described above, they can be orally administered, for example, with an inert diluent or an assimilable edible carrier. The nucleic acid constructs, recombinant cells, and/or pharmaceutical compositions and other ingredients can also be enclosed in a hard or soft shell gelatin capsule, compressed into tablets, or incorporated directly into the individual's diet. For oral therapeutic administration, the active compound can be incorporated with excipients and used in the form of ingestible tablets, buccal tablets, troches, capsules, elixirs, suspensions, syrups, wafers, and the like.

In some embodiments, the nucleic acid constructs of the disclosure can be delivered to a cell or a subject by a lipid-based nanoparticle (LNP). LNP are generally less immunogenic than viral particles. While many humans have preexisting immunity to viral particles there is no pre-existing immunity to LNP. In addition, adaptive immune response against LNP is unlikely to occur which enables repeat dosing of LNP.

Several different ionizable cationic lipids have been developed for use in LNP. These include C12-200, MC3, LN16, and MD1 among others. For example, in one type of LNP, a GalNAc moiety is attached to the outside of the LNP and acts as a ligand for uptake in to the liver via the asialoglycoprotein receptor. Any of these cationic lipids can be used to formulate LNP for delivery of the nucleic acid constructs of the disclosure to the liver.

In some embodiments, a LNP refers to any particle having a diameter of less than 1000 nm, 500 nm, 250 nm, 200 nm, 150 nm, 100 nm, 75 nm, 50 nm, or 25 nm. Alternatively, a nanoparticle can range in size from 1-1000 nm, 1-500 nm, 1-250 nm, 25-200 nm, 25-100 nm, 35-75 nm, or 25-60 nm.

LNPs can be made from cationic, anionic, or neutral lipids. Neutral lipids, such as the fusogenic phospholipid DOPE or the membrane component cholesterol, can be included in LNPs as ‘helper lipids’ to enhance transfection activity and nanoparticle stability. Limitations of cationic lipids include low efficacy owing to poor stability and rapid clearance, as well as the generation of inflammatory or anti-inflammatory responses. LNPs can also have hydrophobic lipids, hydrophilic lipids, or both hydrophobic and hydrophilic lipids.

Any lipid or combination of lipids that have been developed for use in LNP can be used to produce a LNP of the disclosure. Non-limiting examples of lipids suitable for use in production of LNPs include DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Non-limiting examples of cationic lipids suitable for use in production of LNPs include 98N12-5, C12-200, DLin-KC2-DMA (KC2), DLin-MC3-DMA (MC3), XTC, MD1, and 7C1. Non-limiting examples of neutral lipids suitable for use in production of LNPs include DPSC, DPPC, POPC, DOPE, and SM. Non-limiting examples of PEG-modified lipids suitable for use in production of LNPs include PEG-DMG, PEG-CerC14, and PEG-CeraC20.

In some embodiments, the lipids can be combined in any number of molar ratios to produce a LNP. In addition, the polynucleotide(s) can be combined with lipid(s) in a wide range of molar ratios to produce a LNP.

In some embodiments, the therapeutic compositions described herein, e.g., nucleic acid constructs, recombinant cells, and/or pharmaceutical compositions are incorporated into therapeutic compositions for use in methods of preventing or treating a subject who has, who is suspected of having, or who can be at high risk for developing a cancer, an autoimmune disease, and/or an infection.

In some embodiments, the therapeutic compositions described herein, e.g., nucleic acid constructs, recombinant cells, and/or pharmaceutical compositions are incorporated into therapeutic compositions for use in methods of preventing or treating a subject who has, who is suspected of having, or who can be at high risk for developing a microbial infection. In some embodiments, the microbial infection is a bacterial infection. In some embodiments, the microbial infection is a fungal infection. In some embodiments, the microbial infection is a viral infection.

In some embodiments, a composition according to the present disclosure is administered to the subject individually as a single therapy (monotherapy) or as a first therapy in combination with at least one additional therapies (e.g., second therapy). In some embodiments, the second therapy is selected from the group consisting of chemotherapy, radiotherapy, immunotherapy, hormonal therapy, toxin therapy, targeted therapy, and surgery. In some embodiments, the second therapy is selected from the group consisting of chemotherapy, radiotherapy, immunotherapy, hormonal therapy, toxin therapy or surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before the second therapy. In some embodiments, the first therapy is administered after the second therapy. In some embodiments, the first therapy is administered before and after the second therapy. In some embodiments, the first therapy and the second therapy are administered in rotation. In some embodiments, the first therapy and the second therapy are administered together in a single formulation.

### Methods of Producing a Polypeptide of Interest

In an aspect, provided herein are methods for producing a polypeptide of interest, wherein the methods include (i) rearing a transgenic animal of the disclosure; or (ii) culturing a recombinant cell including a nucleic acid construct as disclosed herein under conditions wherein the recombinant cell produces the polypeptide encoded by the srRNA.

Non-limiting exemplary embodiments of the disclosed methods for producing a recombinant polypeptide can include one or more of the following features. In some embodiments, the methods for producing a recombinant polypeptide of the disclosure further include isolating and/or purifying the produced polypeptide. In some embodiments, the methods for producing a polypeptide of the disclosure further include structurally modifying the produced polypeptide to increase half-life.

The discussion of the general methods given herein is intended for illustrative purposes only. Other alternative methods and alternatives will be apparent to those of skill in the art upon review of this disclosure, and are to be included within the spirit and purview of this application.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub combination was individually and explicitly disclosed herein.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purpose.

### EXAMPLES

### Example 1

Swapping of nsP to Functionalize srRNA Vector

An example of a srRNA vector assembly, which unexpectedly resulted in a non-functional SINV AR86 srRNA is as follows. Four ˜4 kb parts were synthesized (Twist Bioscience, Thermo Fisher GeneArt) based on a SINV AR86 reference sequence (Genbank U38305) with a unique restriction enzyme cut site (SpeI, 5′-A′CTAG,T-3′) in place of the coding sequence of the SINV structural genes (where the 5′ A is the next nucleotide after a 3′ adaptor P2A sequence following nucleotide 93 of the structural polyprotein gene, and the 3′ T matches the location of the structural polyprotein's stop codon TGA). A bacteriophage T7 RNA polymerase promoter (5′-TAATACGACTCACTATAG-3′) was included upstream of the SINV genome sequence, and downstream contained a polyA sequence followed by a unique restriction enzyme site (SapI, 5′-GCTCTTC (N)₁′ (N)₃,-3′) followed by a T7 terminator sequence (5′-AACCCCTCTCTAAACGGAGGGGTTTTTTT-3′) followed by a unique restriction enzyme cut site (NotI, 5′-GC′GGCC,GC-3′). The parts were combined in a five-piece Gibson Assembly® reaction (e.g., a restriction-digest linearized plasmid backbone and the four synthesized fragments).

The above vector was functionalized by replacing the SINV AR86 nsP2 with SINV Girdwood nsP2 as follows. The above vector was linearized by restriction digestion with BbvCI and AflII, and the large fragment was isolated by gel extraction following gel electrophoresis. This linearized product lacked part of AR86 nsP1, all of AR86 nsP2, and part of AR86 nsP3. The left insert fragment containing the missing portion of AR86 nsP1 was generated by PCR from the non-functional SINV AR86 srRNA vector template described above. The missing portion of AR86 nsP3 was similarly generated by PCR. The Girdwood nsP2 was generated by PCR from a SINV Girdwood srRNA vector template that was prepared similar to the SINV AR86 srRNA vector above based on the SINV Girdwood reference sequence (Genbank MF459683), using primers that added homologous ends to the other PCR products. The parts were combined in a four-piece Gibson Assembly® reaction (e.g., the original restriction-digest linearized SINV AR86 backbone and the three PCR fragments) to result in a functional SINV AR86 srRNA vector.

Construction of SINV AR86 srRNA vectors containing heterologous genes was carried out as follows: the functional vector described above was linearized by SpeI digestion. The hemagglutinin (HA) gene from influenza (Genbank AY651334) was codon optimized/refactored for human expression in silico and synthesized de novo (IDT). The synthetic product was amplified using primers which added the 5′ and 3′ adaptor sequences to the end of the HA gene. The digestion product and the PCR product were combined by Gibson Assembly® procedure to result in the final vector. Alternatively, the ESR1, PI3K, HER2, and HER3 variants were codon optimized/refactored for human expression in silico and along with the EMCV IRES, were synthesized de novo (GeneArt, IDT). The synthetic products were amplified using primers which added either 5′ and 3′ adaptor sequences to the ends of the genes, or primers which added P2A sequences and/or sequences of homology to neighboring gene inserts. The digestion product and PCR products were combined by Gibson Assembly® procedure to result in the final vector.

### Example 2

Swapping of UTR to Functionalize a srRNA Vector

A further example of a srRNA vector assembly, which unexpectedly resulted in a non-functional CHIKV DRDE-06 srRNA is as follows. Four ˜4 kb parts were synthesized (Twist Bioscience, Thermo Fisher GeneArt) based on a CHIKV DRDE-06 reference sequence (Genbank EF210157) with a unique restriction enzyme cut site (SpeI, 5′-A′CTAG,T-3′) in place of the coding sequence of the CHIKV structural genes (where the 5′ A is the next nucleotide after a 3′ adaptor P2A sequence following nucleotide 93 of the structural polyprotein gene, and the 3′ T matches the location of the structural polyprotein's stop codon TGA). A bacteriophage T7 RNA polymerase promoter (5′-TAATACGACTCACTATAG-3′) was included upstream of the CHIKV genome sequence, and downstream contained a polyA sequence followed by a unique restriction enzyme site (SapI, 5′-GCTCTTC (N)₁′ (N)₃,-3′) followed by a T7 terminator sequence (5′-AACCCCTCTCTAAACGGAGGGGTTTTTTT-3′) followed by a unique restriction enzyme cut site (NotI, 5′-GC′GGCC,GC-3′). The parts were combined in a five-piece Gibson Assembly® reaction (e.g., a restriction-digest linearized plasmid backbone and the four synthesized fragments) to result in the CHIKV DRDE-06 base srRNA vector.

The CHIKV DRDE-06 srRNA vector was functionalized by SpeI and NotI restriction digestion of the non-functional DRDE-06 base vector and was combined in a two-piece Gibson Assembly® reaction with the linearized backbone, and a PCR product from the CHIKV S27 vector containing the 3′ UTR, polyA, and T7 terminator sequence.

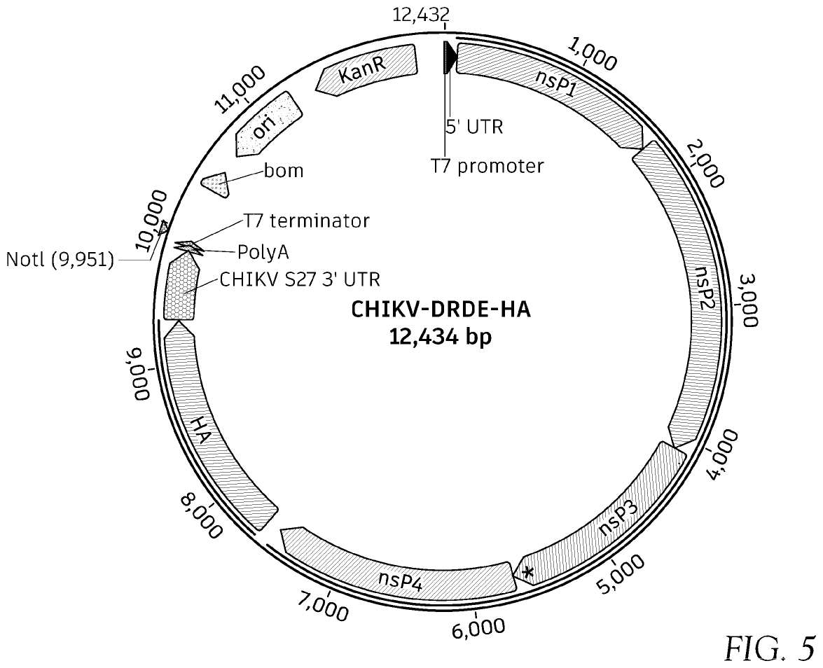

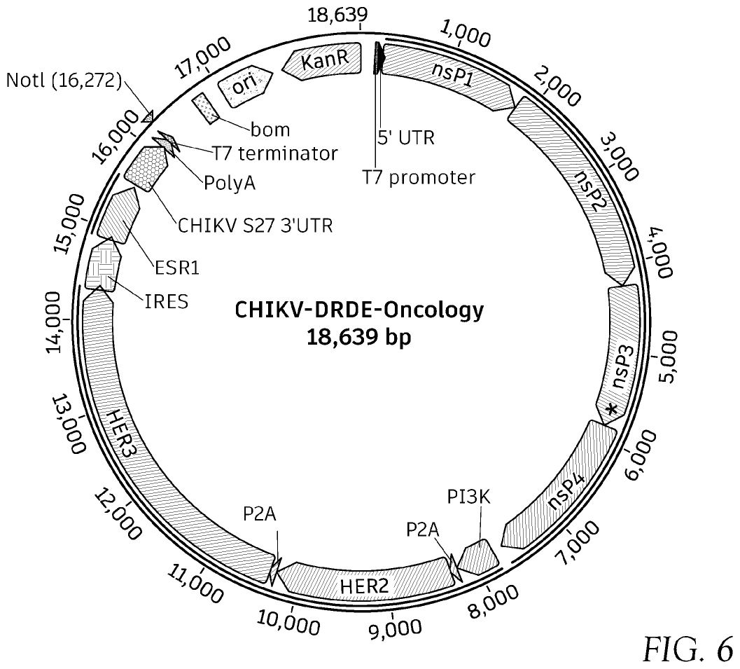

Construction of CHIKV DRDE-06 srRNA vectors containing heterologous genes was carried out as follows: the functional vector described above was linearized by SpeI digestion. The hemagglutinin (HA) gene from influenza (Genbank AY651334) was codon optimized/refactored for human expression in silico and synthesized de novo (IDT). The synthetic product was amplified using primers which added the 5′ and 3′ adaptor sequences to the end of the HA gene. The digestion product and the PCR product were combined by Gibson Assembly® procedure to result in the final vector (CHIKV-DRDE-HA shown in FIG. 5). Alternatively, the ESR1, PI3K, HER2, and HER3 variants were codon optimized/refactored for human expression in silico and along with the EMCV IRES, were synthesized de novo (GeneArt, IDT). The synthetic products were amplified using primers which added either 5′ and 3′ adaptor sequences to the ends of the genes, or primers which added P2A sequences and/or sequences of homology to neighboring gene inserts. The digestion product and PCR products were combined by Gibson Assembly® procedure to result in the final vector (CHIKV-DRDE-Oncology showin in FIG. 6).

### Example 3

### In Vitro Transcription

The functionality of the srRNA generated according to the methods of the disclosure can be assayed using an in vitro transcription procedure, for example, as follows. RNA was prepared by in vitro transcription using a plasmid DNA template that is linearized by enzymatic digestion with SapI, which cuts at the end of the poly(A). Bacteriophage T7 polymerase was used for in vitro transcription with either a 5′ ARCA cap (HiScribe™ T7 ARCA mRNA Kit, NEB) or by uncapped transcription (HiScribe™ T7 High Yield RNA Synthesis Kit, NEB) followed by addition of a 5′ cap 1 (Vaccinia Capping System, mRNA Cap 2′-O-Methyltransferase, NEB). srRNA was purified using phenol/chloroform extraction, lithium chloride precipitation, or column purification (Monarch® RNA Cleanup Kit, NEB). RNA concentration is determined by absorbance at 260 nm (Nanodrop, Thermo Fisher Scientific). Identity of srRNA products are evaluated by gel electrophoresis.

### Example 4

In Vitro Evaluation of Functional srRNAs

This Example describes the results of in vitro experiments performed to evaluate functionality of constructs described in Example 1 above.

In vitro transcription: srRNA was prepared by in vitro transcription using a plasmid DNA template linearized by enzymatic digestion. In these examples, the DNA was either linearized with NotI, which cuts downstream of the T7 terminator, or linearized with SapI, which cuts at the end of the poly(A). Bacteriophage T7 polymerase was used for in vitro transcription with either a 5′ ARCA cap (HiScribe™ T7 ARCA mRNA Kit, NEB) or by uncapped transcription (HiScribe™ T7 High Yield RNA Synthesis Kit, NEB) followed by addition of a 5′ cap 1 (Vaccinia Capping System, mRNA Cap 2′-O-Methyltransferase, NEB). srRNA was purified using phenol/chloroform extraction, lithium chloride precipitation, or column purification (Monarch® RNA Cleanup Kit, NEB). RNA concentration was determined by absorbance at 260 nm (Nanodrop, Thermo Fisher Scientific).

Replication. srRNA was transformed by electroporation into BHK-21 or Vero cells (e.g. 4D-Nucleofector™, Lonza). At 17-20 h following transformation, the cells were fixed and permeabilized (eBioscience™ Foxp3/Transcription Factor Staining Buffer Set, Invitrogen) and stained using a PE-conjugated anti-dsRNA mouse monoclonal antibody (J2, Scicons) to quantify the frequency of dsRNA+ cells and the mean fluorescence intensity (MFI) of dsRNA in individual cells by fluorescence flow cytometry.

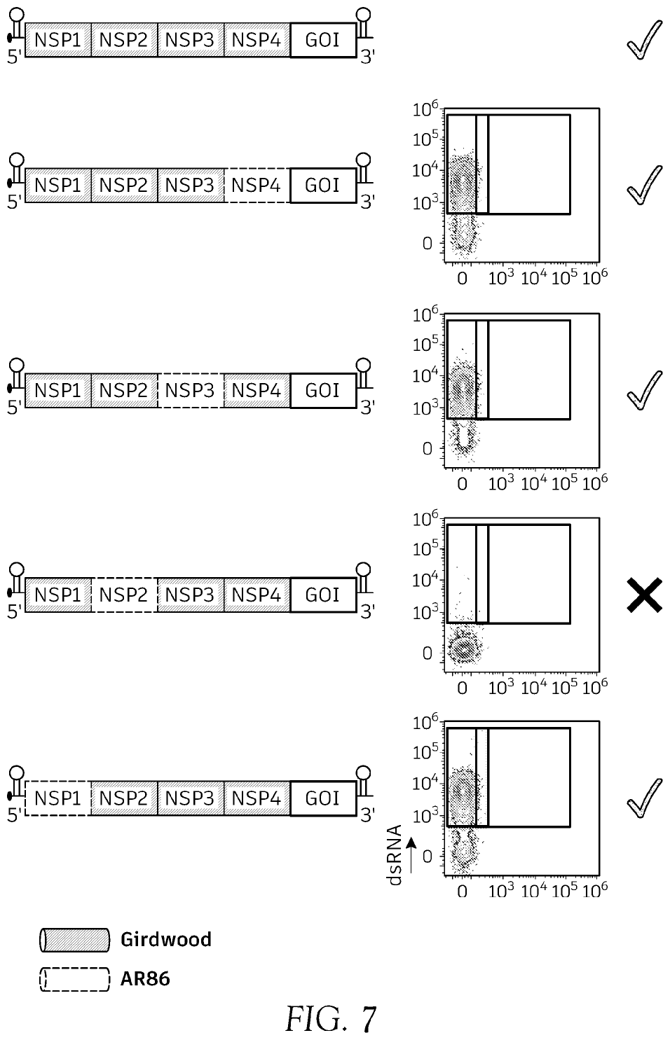

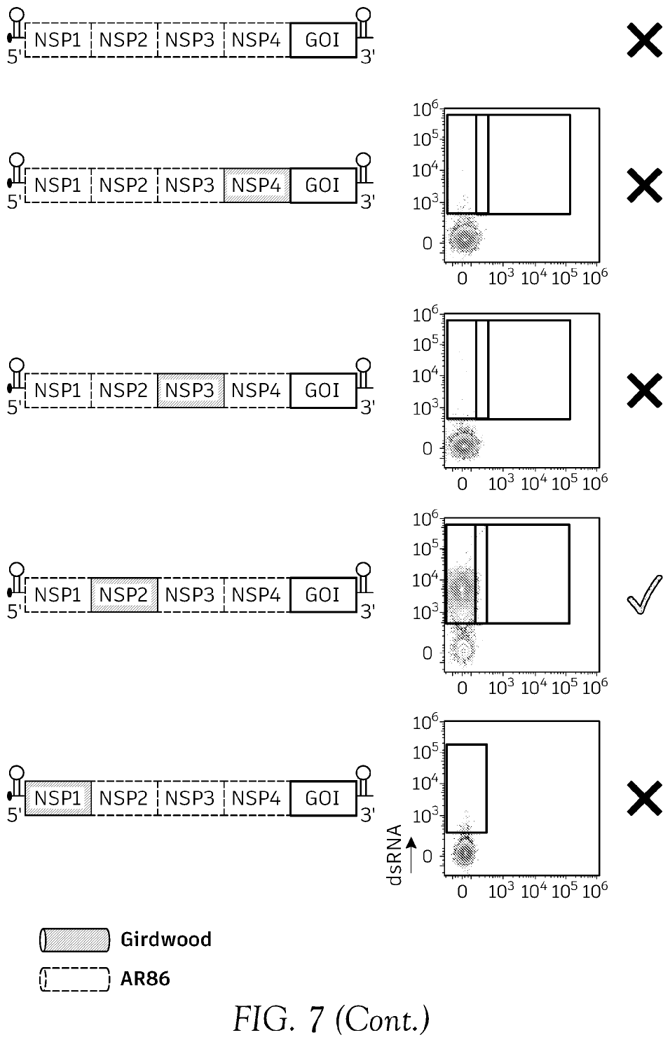

As shown in FIG. 7, a non-functional SINV strain AR86 srRNA can be functionalized by using a heterologous nsP2 from SINV strain Girdwood. This is demonstrated by the increase in the frequency of dsRNA+ cells when the heterologous nsP2 from SINV strain Girdwood is used.

### Example 5

In Vivo Evaluation of Functional srRNAs

This Example describes the results of in vivo experiments performed to evaluate any differential immune responses following vaccination with a functionalized srRNA constructs described in Example 1 above (e.g., both unformulated and LNP formulated vectors).

Mice and injections. Female C57BL/6 or BALB/c mice were purchased from Charles River Labs or Jackson Laboratories. On day of dosing, between 0.1-10 μg of material was injected intramuscularly split into both quadricep muscles. Vectors were administered either unformulated in saline, or LNP-formulated. Animals were monitored for body weight and other general observations throughout the course of the study. For immunogenicity studies, animals were dosed on Day 0 and Day 21. Spleens were collected at Day 35, and serum was isolated at Days 0, 14, and 35. For protein expression studies, animals are dosed on Day 0, and bioluminescence is assessed on Days 1, 3, and 7. In vivo imaging of luciferase activity is done using an IVIS system at the indicated time points.

LNP formulation. srRNA was formulated in lipid nanoparticles using a microfluidics mixer and analyzed for particle size, polydispersity using dynamic light scattering and encapsulation efficiency. Molar ratios of lipids used in formulating LNP particles is 30% C12-200, 46.5% Cholesterol, 2.5% PEG-2K and 16% DOPE.

ELISpot. To measure the magnitude of Influenza-specific T cell responses, IFNγ ELISpot analysis was performed using Mouse IFNγ ELISpot PLUS Kit (HRP) (MabTech) as per manufacturer's instructions. In brief, splenocytes were isolated and resuspended to a concentration of 5×10⁶ cells/mL in media containing peptides representing either CD4+ or CD8+ T cell epitopes to HA, PMA/ionomycin as a positive control, or DMSO as a mock stimulation.

Intracellular cytokine staining. Spleens are isolated according to the methods outlined for ELISpots, and 1×10⁶ cells are added to cells containing media in a total volume of 200 μL per well. Each well contains peptides representing either CD4+ or CD8+ T cell epitopes to HA, PMA/ionomycin as a positive control, or DMSO as a mock stimulation. After 1 hour, GolgiPlug™ protein transport inhibitor (BD Biosciences) is added to each well. Cells are incubated for another 5 hours. Following incubation, cells are surface stained for CD8+ (53-6.7), CD4+ (GK1.5), B220 (B238128), Gr-1 (RB6-8C5), CD16/32 (M93) using standard methods. Following surface staining, cells are fixed and stained for intracellular proteins as per standard methods for IFNγ (RPA-T8), IL-2 (JES6-5H4), and TNF (MP6-XT22). Cells are then subsequently analyzed on a flow cytometer and the acquired FCS files analyzed using FlowJo software version 10.4.1.

Antibodies. Antibody responses to measure total HA-specific IgG were measured using ELISA kits from Alpha Diagnostic International as per manufacturer's instructions.

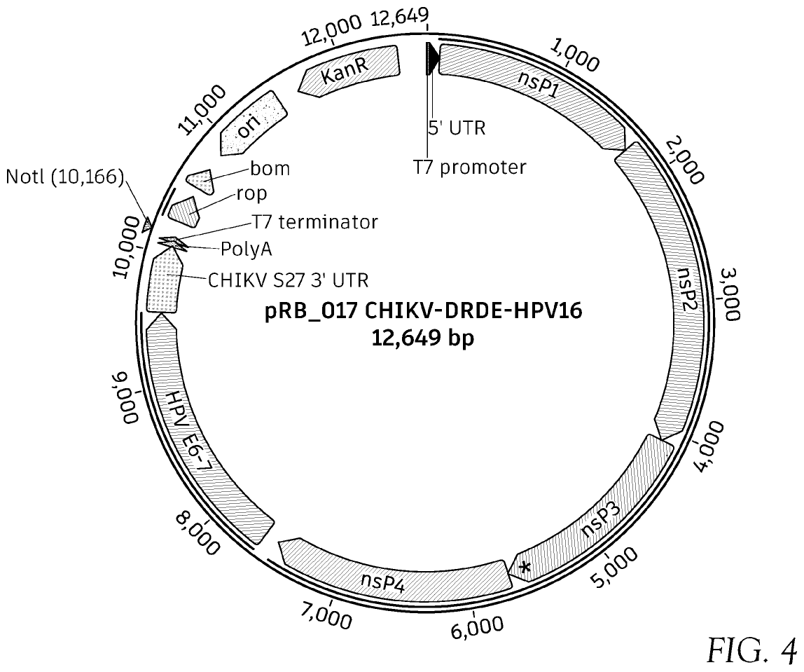

A CHIKV-DRDE-HPV16 construct (shown in FIG. 4) was generated using the methods described in Example 2 above. The construct was LNP formulated and injected into mice and evaluated by ELISpot assay as described above.

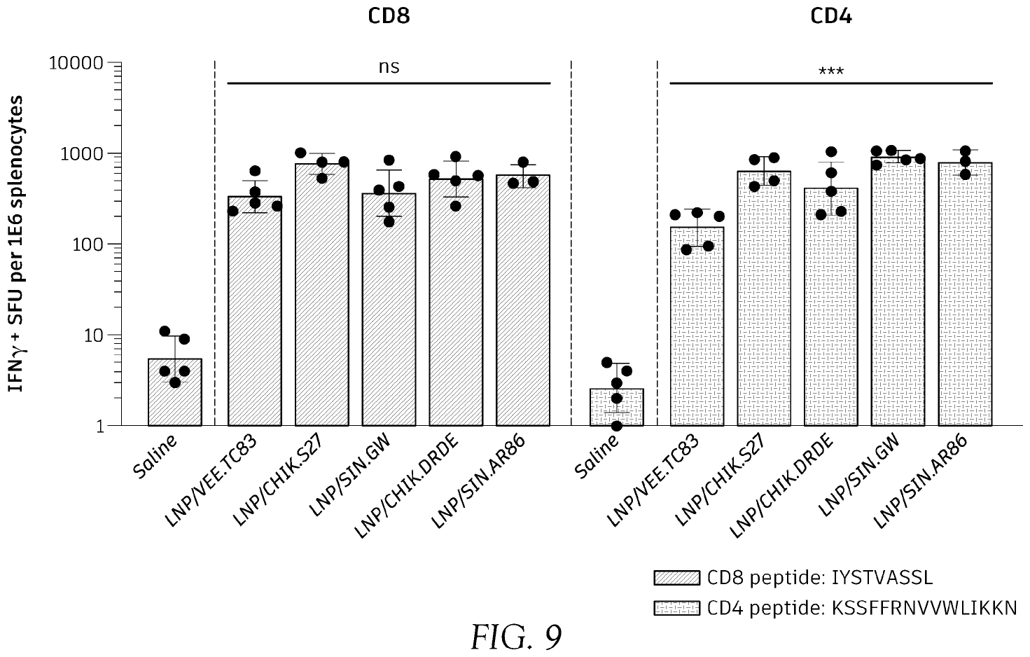

A SINV-AR86-HA construct and a CHIKV-DRDE-HA construct (shown in FIG. 5), were generated using the methods described in Examples 1 and 2 above. The constructs were LNP formulated and injected into mice and evaluated by ELISPot assay as described above. The results demonstrate that these functionalized srRNA vectors can produce immune responses in vivo (FIG. 9).

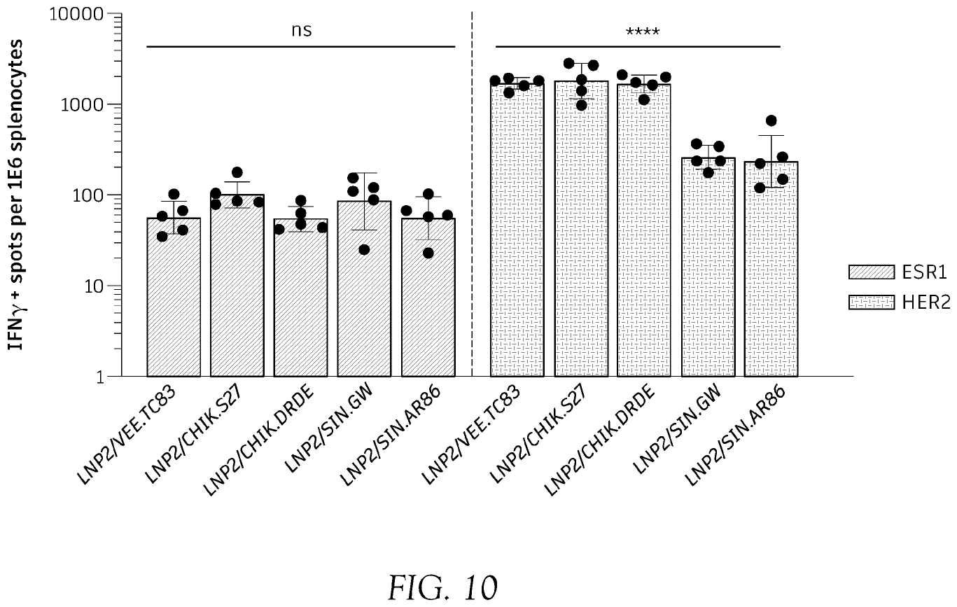

Similarly, a SINV-AR86-Oncology construct and a CHIKV-DRDE-Oncology construct (shown in FIG. 6), were generated using the methods described in Examples 1 and 2 above. The constructs were LNP formulated and injected into mice and evaluated by ELISPot assay with ESR1 and HER2 peptides (as opposed to HA peptides.) The results demonstrate that these functionalized srRNA vectors can produce immune responses in vivo (FIG. 10).

### Example 6

In Vivo Evaluation of Functional srRNA Vectors with Heterologous Nonstructural Protein Genes or with Heterologous UTRs

This Example describes the results of in vivo experiments performed to evaluate any differential immune responses following vaccination with functionalized srRNA constructs derived from srRNA vectors described in Example 1 and 2 above.

In these experiments, synthetic srRNA constructs derived from Venezuelan equine encephalitis virus (VEE.TC83), Chikungunya virus strains S27 (CHIK.S27) and DRDE-06 (CHIK.DRDE), Sindbis virus strains Girdwood (SIN.GW) and AR86 (SIN.AR86), and Eastern equine encephalitis virus (EEE.FL93) were designed and subsequently evaluated.

Mice and injections. Female BALB/c mice were purchased from Charles River Labs or Jackson Laboratories. On day of dosing, between 0.15-1.5 μg of material was injected intramuscularly split into both quadricep muscles. Vectors were LNP-formulated. Animals were monitored for body weight and other general observations throughout the course of the study. For immunogenicity studies, animals were dosed on Day 0 and Day 21. Spleens were collected at Day 14 and/or 35, and serum was isolated at Days 14, and/or 35.

LNP formulation. srRNA was formulated in lipid nanoparticles (LNPs) using a microfluidics mixer and analyzed for particle size, polydispersity using dynamic light scattering and encapsulation efficiency. LNPs are composed of an ionizable lipid, cholesterol, PEG-2K, and DOPE.

ELISpot. To measure the magnitude of antigen-specific T cell responses, IFNγ ELISpot analysis was performed using Mouse IFNγ ELISpot PLUS Kit (HRP) (MabTech) as per manufacturer's instructions. In brief, splenocytes are isolated and resuspended to a concentration of 1-5×10⁶ cells/mL in media containing either peptide pools corresponding to rabies virus glycoprotein G, PMA/ionomycin as a positive control, or DMSO as a mock stimulation.

Antibodies. Neutralizing antibody responses to rabies virus were measured using the Rapid Fluorescent Focus Inhibition Test. In brief, serum dilutions were mixed with a standard amount of live rabies virus and incubated. If neutralizing anti-rabies antibodies are present, they will neutralize the virus. Next, cultured cells were added and the serum/virus/cells were incubated together. Uncoated rabies virus (i.e. that has not been neutralized by antibodies), will infect the cells and this can be visualized by microscopy. Calculation of the endpoint titer was made from the percent of virus infected cells observed on the slide.

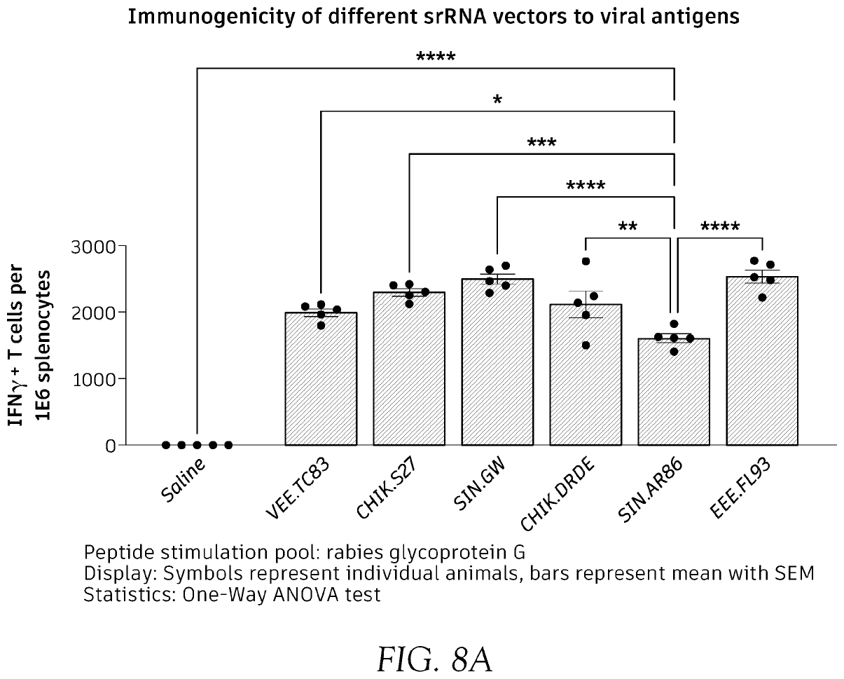

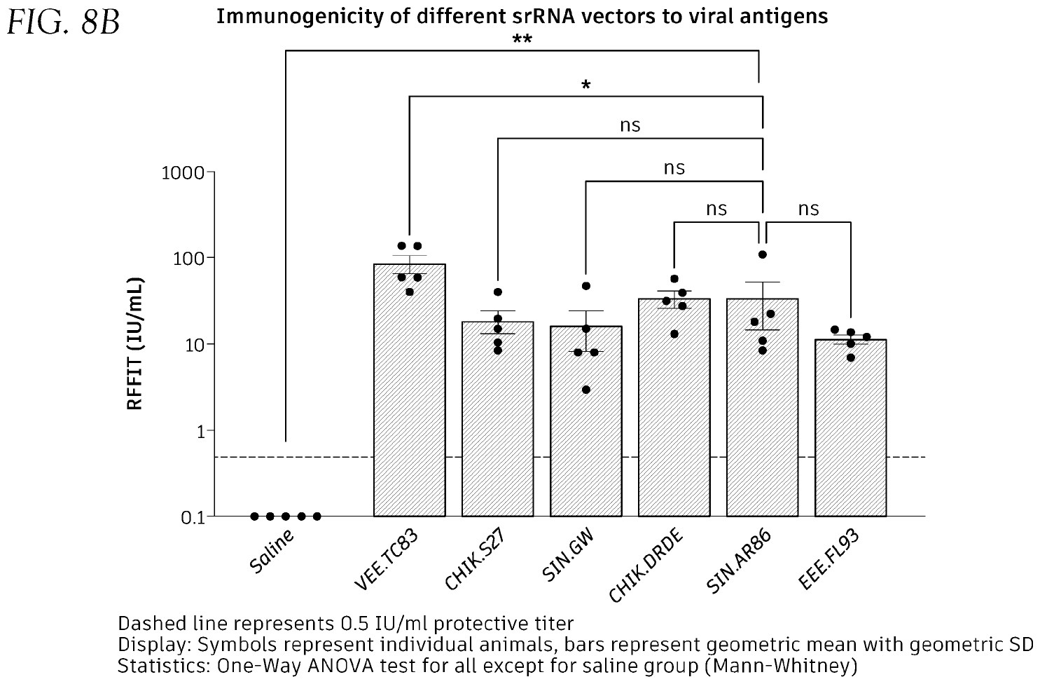

In vivo immunogenicity of a plurality of functionalized srRNAs encoding a viral antigen, rabies virus glycoprotein G, was assessed by evaluating antigen-specific splenic T cell responses by ELISpot (FIG. 8A) and anti-rabies neutralizing antibody titers from sera (FIG. 8B) after two immunizations. All srRNA-immunized groups showed robust T cell responses compared to saline controls (FIG. 8A), but differential responses were observed between srRNA vaccines. Similarly, all srRNA-immunized groups showed protective neutralizing antibody titers with some variations between srRNA vaccines (FIG. 8B).

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A method for generating a functional self-replicating RNA (srRNA), the method comprising:
(a) providing one or more positive-sense single-stranded RNA (+ssRNA) viral genomes or non-functional srRNAs, wherein at least one of the one or more +ssRNA viral genomes or srRNAs is non-functional;
(b) removing one or more RNA polymerase termination sites or an RNA polymerase cryptic termination sites from the one or more +ssRNA viral genomes or srRNAs;
(c) generating a plurality of nucleic acid fragments each comprising a nucleotide sequence derived from the one or more +ssRNA viral genomes or srRNAs; and
(d) assembling the plurality of nucleic acid fragments to generate a de novo functional srRNA assembly.

2. The method of claim 1, wherein the one or more RNA polymerase termination sites or cryptic termination sites comprises (i) a bacteriophage T7 termination site or a cryptic T7 termination site; and/or (ii) an SP6 RNA polymerase termination site or an SP6 RNA polymerase cryptic termination site.

3. The method of any one of claims 1 to 2, wherein the plurality of nucleic acid fragments are each about 60 nucleotides to about 5,000 nucleotides in length.

4. The method of any one of claims 1 to 3, wherein the plurality of nucleic acid fragments are single-stranded or double-stranded nucleic acids.

5. The method of any one of claims 1 to 4, wherein the de novo functional srRNA assembly is devoid of at least a portion of the nucleic acid sequence encoding one or more viral structural proteins.

6. The method of any one of claims 1 to 5, wherein the de novo functional srRNA assembly is devoid of the nucleic acid sequence encoding one or more viral structural proteins.

7. The method of any one of claims 1 to 6, wherein the de novo functional srRNA assembly is devoid of a substantial portion of the nucleic acid sequence encoding one or more viral structural proteins.

8. The method of any one of claims 1 to 7, wherein the de novo functional srRNA assembly comprises no nucleic acid sequence encoding viral structural proteins.

9. The method of any one of claims 1 to 8, wherein at least one of the one or more +ssRNA viral genomes or srRNAs is of a virus belonging to the Alphavirus genus of the Togaviridae family.

10. The method of any one of claims 1 to 9, wherein at least one of the one or more +ssRNA viral genomes or srRNAs is of an alphavirus species belonging to VEEV/EEEV group, or the SFV group, or the SINV group.

11. The method of any one of claims 1 to 10, wherein at least one of the +ssRNA viral genomes or srRNAs is of an alphavirus selected from Eastern equine encephalitis virus (EEEV), Venezuelan equine encephalitis virus (VEEV), Everglades virus (EVEV), Mucambo virus (MUCV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu virus (NDUV), Madariaga virus (MADV), and Buggy Creek virus.

12. The method of any one of claims 1 to 10, wherein at least one of the +ssRNA viral genomes or srRNAs is of Eastern equine encephalitis virus (EEEV), Chikungunya virus (CHIKV), Sindbis virus (SINV), Venezuelan equine encephalitis virus (VEE), Madariaga virus (MADV), Western equine encephalitis virus (WEEV), or Semliki Forest virus (SFV).

13. The method any one of claims 1 to 12, wherein the method further comprises incorporating a nucleic acid sequence encoding a heterologous gene into the de novo srRNA assembly.

14. The method of claim 13, wherein the heterologous gene is operably linked to a subgenomic (sg) promoter.

15. The method of claim 14, wherein the sg promoter is a 26S subgenomic promoter.

16. The method of any one of claims 1 to 15, wherein the method further comprises removing one or more restriction enzyme sites from one or more of the +ssRNA genomes or srRNAs.

17. The method of claim 16, wherein at least one of the removed restriction enzyme sites is recognized by a restriction enzyme suitable for linearization of the de novo srRNA assembly or for insertion of the heterologous gene into the de novo srRNA assembly.

18. The method of any one of claims 1 to 17, wherein the generated functional srRNA assembly comprises a 3′ polyadenylate tract (poly(A) tail).

19. The method of claim 18, wherein the 3′ poly(A) tail comprises at least 11 adenine nucleotides.

20. The method of any one of claims 1 to 19, wherein the method further comprises replacing one or more untranslated regions (UTR) in the de novo srRNA assembly with a UTR from a different species, subspecies, or strain of the +ssRNA viral genome.

21. The method of claim 20, wherein the method comprises replacing a 3′ UTR.

22. The method of claim 20, wherein the method comprises replacing a 5′ UTR.

23. The method of claim 20, wherein the method comprises replacing a 3′UTR and a 5′ UTR.

24. The method of any one of claims 20 to 23, wherein the method further comprises selecting the UTR from a virulent species or an avirulent species of a +ssRNA virus.

25. The method of any one of claims 1 to 24, wherein the method further comprises replacing a non-structural protein (nsP), or a portion thereof, in the de novo srRNA assembly with a heterologous nsP.

26. The method of claim 25, wherein the heterologous nsP or portion thereof is from another +ssRNA virus species, subspecies, or strain.

27. The method of claim 25, wherein the nsP or portion thereof is derived from another strain of the same +ssRNA virus species.

28. The method of any one of claims 25 to 27, wherein the nsP or portion thereof is nsP1, nsP2, nsP3, nsP4, or a portion of any thereof.

29. The method of any one of claims 25 to 28, wherein the method further comprises selecting the nsP from a virulent species or an avirulent species of a +ssRNA virus.

30. The method of any one of claims 1 to 29, wherein the method further comprises assessing functionality of the de novo srRNA assembly.

31. The method of claim 30, wherein the assessing functionality is carried out in vitro, in vivo, and/or ex vivo.

32. The method of claim 30 or 31, wherein the assessing functionality comprises analyzing the de novo srRNA assembly for capability of self-replicating in vivo and/or ex vivo.

33. The method of any one of claims 30 to 32, wherein the assessing functionality comprises an assay selected from the group consisting of: detection of RNA replication, detection of viral protein expression, detection of cytopathic effect (CPE), and detection of heterologous gene expression.

34. The method of any one of claims 30 to 33, wherein the assessing functionality of the de novo srRNA assembly does not comprise incorporating a nucleic acid sequence encoding a heterologous gene into the de novo srRNA assembly.

35. A functional self-replicating RNA (srRNA) generated by a method according to any one of claims 1 to 34, wherein the functional srRNA comprises a heterologous UTR and/or a heterologous nsP.

36. A nucleic acid construct encoding the srRNA according to claim 35.

37. A vector comprising the nucleic acid construct of claim 36.

38. A recombinant cell comprising:
a) a functional srRNA according to claim 35;
b) a nucleic acid construct according to claim 36; and/or
c) a vector according to claim 37.

39. The recombinant cell of claim 38, wherein the recombinant cell is a eukaryotic cell.

40. The recombinant cell of claim 39, wherein the recombinant cell is an animal cell.

41. The recombinant cell of claim 40, wherein the animal cell is a vertebrate animal cell or an invertebrate animal cell.

42. The recombinant cell of claim 40, wherein the animal cell is an insect cell.

43. The recombinant cell of claim 42, wherein the insect cell is a mosquito cell.

44. The recombinant cell of claim 38, wherein the recombinant cell is a mammalian cell.

45. The recombinant cell of claim 44, wherein the recombinant cell is selected from the group consisting of a monkey kidney CV1 cell transformed by SV40 (COS-7), a human embryonic kidney cell (e.g., HEK 293 or HEK 293 cell), a baby hamster kidney cell (BHK), a mouse sertoli cell (e.g., TM4 cells), a monkey kidney cell (CV1), a human cervical carcinoma cell (HeLa), canine kidney cell (e.g., MDCK), buffalo rat liver cell (e.g., BRL 3A), human lung cell (e.g., W138), human liver cell (e.g., Hep G2), mouse mammary tumor (MMT 060562), TRI cell, FS4 cell, a Chinese hamster ovary cell (CHO cell), an African green monkey kidney cell (e.g., Vero cell), a human A549 cell, a human cervix cell, a human CHME5 cell, a human PER.C6 cell, a NS0 murine myeloma cell, a human epidermoid larynx cell, a human fibroblast cell, a human HUH-7 cell, a human MRC-5 cell, a human muscle cell, a human endothelial cell, a human astrocyte cell, a human macrophage cell, a human RAW 264.7 cell, a mouse 3T3 cell, a mouse L929 cell, a mouse connective tissue cell, a mouse muscle cell, and a rabbit kidney cell.

46. A pharmaceutical composition comprising:
a) a functional srRNA according to claim 35; or
b) a nucleic acid construct according to claim 36;
c) a vector according to claim 37; and/or
c) a recombinant cell according to any of claims 38 to 45.

47. The pharmaceutical composition of claim 46, comprising a functional srRNA of claim 30, and a pharmaceutically acceptable excipient.

48. The pharmaceutical composition of claim 46, comprising a nucleic acid construct of claim 31, and a pharmaceutically acceptable excipient.

49. The pharmaceutical composition of claim 46, comprising a vector of claim 32, and a pharmaceutically acceptable excipient.

50. The pharmaceutical composition of claim 46, comprising a recombinant cell of any one of claims 38 to 45, and a pharmaceutically acceptable excipient.

51. The pharmaceutical composition of any one of claims 46 to 50, wherein the composition is formulated in a liposome, a lipid-based nanoparticle (LNP), a polymer nanoparticle, a polyplex, a viral replicon particle (VRP), a microsphere, an immune stimulating complex (ISCOM), a conjugate of a bioactive ligand, or a combination of any thereof.

52. The pharmaceutical composition of any one of claims 46 to 51, wherein the composition is an immunogenic composition.

53. The pharmaceutical composition of claim 52, wherein the immunogenic composition is formulated as a vaccine.

54. The pharmaceutical composition of any one of claims 46 to 51, wherein the composition is substantially non-immunogenic to a subject.

55. The pharmaceutical composition of any one of claims 46 to 52, wherein the pharmaceutical composition is formulated as an adjuvant.

56. The pharmaceutical composition of any one of claims 46 to 55, wherein the pharmaceutical composition is formulated for one or more of intranasal administration, transdermal administration, intraperitoneal administration, intramuscular administration, intratracheal administration, intranodal administration, intratumoral administration, intraarticular administration, intravenous administration, subcutaneous administration, intravaginal administration, intraocular, rectal, and oral administration.

57. A kit for inducing a pharmacodynamic effect, producing polypeptide of interest, for the prevention, and/or for the treatment of a health condition, the kit comprising:
a) a functional srRNA according to claim 35;
b) a nucleic acid construct according to claim 36;
c) a vector according to claim 37;
d) a recombinant cell of any one of claims 38 to 45; and/or
e) a pharmaceutical composition of any one of claims 46 to 56.

58. A transgenic animal comprising:
a) a functional srRNA according to claim 5;
b) a nucleic acid construct according to claim 36;
c) a vector according claim 37; and/or
d) a recombinant cell of any one of claims 38 to 45.

59. The transgenic animal of claim 58, wherein the animal is a vertebrate animal or an invertebrate animal.

60. The transgenic animal of claim 58, wherein the animal is an insect.

61. The transgenic animal of claim 58, wherein the animal is a mammal.

62. The transgenic animal of claim 61, wherein the mammal is a non-human mammal.

63. A method for producing a polypeptide of interest, comprising (i) rearing a transgenic animal according to any one of claims 58 to 62, or (ii) culturing a recombinant cell comprising a nucleic acid construct according to claim 36 under conditions wherein the recombinant cell produces the polypeptide encoded by the srRNA.

64. A method for inducing a pharmacodynamic effect in a subject, the method comprises administering to the subject a composition comprising:
(a) a functional srRNA of claim 35;
(b) a nucleic acid construct of claim 36;
(c) a recombinant cell of any one of claims 38 to 45; and/or
(d) a pharmaceutical composition of any one of claims 46 to 56.

65. The method of claim 64, wherein the pharmacodynamic effect comprises eliciting an immune response in the subject.

66. A method for preventing or treating a health condition in a subject, the method comprises administering to the subject a composition comprising:
(a) a functional srRNA of claim 350;
(b) a nucleic acid construct of claim 36;
(c) a recombinant cell of any one of claims 38 to 45; and/or
(d) a pharmaceutical composition of any one of claims 46 to 56.
